(19) United States
(12) Patent Application Publication
(10) Pub. No.: US 2026/0335436 A1
(43) Pub. Date: Oct. 29, 2026

(54) COMPOSITION FOR PREVENTING OR TREATING FIBROSIS COMPRISING NATURAL KILLER (NK) CELLS

(71) Applicant: KOREA RESEARCH INSTITUTE OF BIOSCIENCE AND BIOTECHNOLOGY, Daejeon (KR)

(72) Inventors: Yee Sook CHO, Daejeon (KR); Binna SEOL, Daejeon (KR); Han-Seop KIM, Daejeon (KR); Jae Yun KIM, Daejeon (KR)

(73) Assignee: KOREA RESEARCH INSTITUTE OF BIOSCIENCE AND BIOTECHNOLOGY, Daejeon (KR)

(21) Appl. No.: 19/700,484

(22) Filed: Jun. 8, 2026

Related U.S. Application Data
(63) Continuation-in-part of application No. PCT/KR2024/014127, filed on Sep. 19, 2024.

(30) Foreign Application Priority Data
Dec. 7, 2023 (KR) 10-2023-0176905

(51) Int. Cl. A61K 40/15; A61K 40/30; A61K 40/35; A61P 19/04

(52) U.S. Cl. CPC A61K 40/15; A61K 40/30; A61K 40/35; A61P 19/04

(57) ABSTRACT

The present invention relates to a pharmaceutical composition and a cell therapy agent for preventing or treating fibrosis, comprising directly reprogrammed natural killer (drNK) cells prepared by reprogramming isolated cells. Specifically, the present invention relates to a pharmaceutical composition and a cell therapy agent for preventing or treating a fibrotic disease, comprising drNK cells expressing one or more selected from the group consisting of CD56^{superbright}, CD16^{superbright}, and a combination thereof.

### CROSS-REFERENCE TO RELATED APPLICATIONS

This application is a bypass continuation-in-part application of Patent Application No. PCT/KR2024/014127 filed Sep. 19, 2024, which claims priority to Korean Patent Application No. 10-2023-0176905 filed Dec. 7, 2023, the entire disclosures of which are incorporated herein by reference.

### TECHNICAL FIELD

The present invention relates to a pharmaceutical composition and a cell therapy agent for preventing or treating fibrosis, and specifically relates to a composition comprising directly reprogrammed natural killer (drNK) cells prepared by reprogramming isolated cells. In addition, the drNK cells are characterized by expressing CD56^{superbright}, CD16^{superbright}, or a combination thereof.

### BACKGROUND ART

Fibrosis is a pathological condition characterized by excessive accumulation of fibrous connective tissue, particularly collagen, in an organ or tissue, resulting in thickening and stiffening of the affected area. This process occurs due to chronic inflammation or injury in which normal tissue is replaced by scar tissue, impairing the structure and function of the affected organ. Fibrosis can occur in various organs such as the lungs, liver, kidneys, heart, and skin, and is often associated with progressive, irreversible damage, contributing to the development of chronic diseases.

In the lungs, conditions such as idiopathic pulmonary fibrosis (IPF) cause lung tissue to thicken and scar, resulting in progressive respiratory failure. As seen in liver cirrhosis and non-alcoholic steatohepatitis (NASH), where extensive scarring occurs due to chronic damage, the liver can also be affected. In the kidneys, chronic kidney disease (CKD) and glomerulonephritis can cause fibrosis, which impairs kidney function and potentially leads to renal failure. Cardiac fibrosis often occurs after a heart attack or chronic hypertension, resulting in stiffening of the cardiac muscle and impaired function. Systemic sclerosis or scleroderma is an autoimmune disease that causes extensive fibrosis and affects the skin and internal organs. In the intestine, chronic inflammatory diseases such as Crohn's disease can lead to fibrous strictures and cause intestinal obstruction. Cystic fibrosis is a genetic disease that primarily affects various organs of the body, such as the lungs, liver, pancreas, urinary system, reproductive system, and sweat glands, in which thick mucus is produced, causing persistent infections and fibrosis. Dupuytren's contracture is a condition characterized by permanent contraction of fibrous tissue in the finger joints in which the fascial tissue between the palm skin and tendons becomes abnormally thickened, preventing the fingers from spreading. As seen in retinal fibrosis and conjunctival fibrosis, fibrosis can also affect the eyes and often occurs due to chronic inflammation or injury. In the skin, fibrous conditions such as keloids and hypertrophic scars can develop following an injury, causing the scar to thicken and swell. Chronic pancreatitis can induce fibrosis in the pancreas, impairing its function and potentially leading to complications such as diabetes. Myelofibrosis, which occurs in conditions such as primary myelofibrosis (PMF) that develops spontaneously due to specific genetic mutations, affects blood cell production by replacing healthy bone marrow tissue with fibrous tissue.

Many existing treatments target symptoms or underlying conditions rather than directly addressing fibrosis itself, resulting in limited efficacy, and are primarily effective in slowing disease progression rather than reversing the fibrotic process. These treatments can have serious side effects and, given the complex and multifactorial nature of fibrosis, may not be effective for all types of fibrotic diseases or all patients. Furthermore, the lack of biomarkers for early detection and monitoring makes timely and accurate treatment difficult, presenting challenges in effectively managing the disease. Currently, active development of therapies focused on disrupting key pathways associated with fibrosis is underway, including pathway (such as TGF-β and Wnt signaling)-specific inhibitors, antifibrotic small molecules and biologics, cell-based therapies such as mesenchymal stem cells, and gene therapies. However, there is an increasing demand for the development of more targeted, safe, and effective therapies capable of stopping or even reversing fibrosis in various organs.

Myofibroblasts, known as the fibrosis-causing cells in various tissues and organs, are important for wound healing and tissue repair. In the context of fibrosis, they become overactivated, resulting in the excessive production of extracellular matrix (ECM) components (including collagen) and the accumulation of fibrous tissue, which in turn causes stiffening and dysfunction of the affected organs. Myofibroblasts are known to differentiate from various progenitor cells, such as fibroblasts, perivascular cells, hepatic stellate cells, and epithelial cells, through pro-fibrotic stimuli such as activation of TGF-β signaling, mechanical stress, and inflammatory cytokines. In animal models of fibrosis, the presence of myofibroblasts in fibrotic lesions is known to be associated with the onset of active fibrosis, and the persistence and localization of myofibroblasts in fibrotic lesions in human disease are known to be associated with disease progression (Kuhn C. et al. Am J Pathol. 1991; 138(5): 1257-65). Accordingly, there is growing interest in the development of fibrosis treatments targeting myofibroblasts, such as suppressing the activation and differentiation of myofibroblasts or promoting apoptosis, as strategies to disrupt the fibrotic process and reduce the burden of fibrotic diseases in order to develop more effective and targeted antifibrosis therapies.

Meanwhile, natural killer (NK) cells, which have the ability to eliminate activated myofibroblasts, are emerging as a promising research target for the development of fibrosis treatments. Although studies using cytokines such as IL-2, IL-15, and IL-18 to enhance the cytotoxic activity of existing NK cells against myofibroblasts have been reported, the low antifibrotic efficacy and difficulties in producing therapeutic NK cells remain as issues requiring improvement.

Recently, with advances in direct reprogramming technology, the development of techniques for directly producing functional cells with high clinical utility without undergoing stem cell production and differentiation processes has been actively underway. Functional cells produced through direct reprogramming techniques are increasingly recognized for their technical advantages, including low risks of epigenetic remodeling and tumorigenesis, and the ease of enhancing safety, reliability, and efficiency by simplifying the cell production process. Such characteristics are expected to ultimately contribute to overcoming barriers to commercialization by drastically shortening and reducing the time and cost required for therapeutic development. Accordingly, research and development efforts to secure raw materials for cell therapies targeting various diseases using direct reprogramming techniques are continuously increasing.

To date, there have been no reports on the preventive, therapeutic, and ameliorating effects of NK cells produced by direct reprogramming on fibrosis.

### DISCLOSURE

### Technical Problem

The present invention aims to provide a technique that is differentiated from existing techniques for obtaining natural killer cells through stem cell production and differentiation processes.

Specifically, the present invention aims to provide a method and a composition for preparing directly reprogrammed natural killer (drNK) cells from somatic cells, which are relatively easy to obtain, through direct reprogramming, and for utilizing the same in the prevention or treatment of fibrotic diseases.

### Technical Solution

One object of the present invention is to provide a pharmaceutical composition for preventing or treating a fibrotic disease, comprising directly reprogrammed natural killer (drNK) cells expressing one or more selected from the group consisting of CD56^{superbright}, CD16^{superbright}, and a combination thereof.

Another object of the present invention is to provide a cell therapy composition for preventing or treating fibrosis, comprising directly reprogrammed natural killer (drNK) cells expressing one or more selected from the group consisting of CD56^{superbright}, CD16^{superbright}, and a combination thereof.

Another object of the present invention is to provide a method for preventing or treating fibrosis, comprising administering to a subject suspected of having fibrosis, excluding humans, a pharmaceutical composition for preventing or treating a fibrotic disease, comprising directly reprogrammed natural killer (drNK) cells expressing one or more selected from the group consisting of CD56^{superbright}, CD16^{superbright}, and a combination thereof.

### Advantageous Effects

It was confirmed that directly reprogrammed natural killer (drNK) cells of the present invention expressing one or more selected from the group consisting of CD56^{superbright}, CD16^{superbright}, and a combination thereof exhibit a superior direct killing ability compared to control natural killer cells known in the art when treated against hepatic stellate cells (hepatic fibrosis cell model), alveolar epithelial cells (pulmonary fibrosis cell model), cardiac cells (cardiac fibrosis cell model), skin cells (dermal fibrosis cell model), and renal cells (renal fibrosis cell model), which are fibrosis-causing cells associated with the induction of fibrotic responses in each respective tissue. Furthermore, when conditioned medium from the drNK cells was applied to the fibrosis cell models above, collagen production, activity, and viability thereof were significantly reduced compared to that from control natural killer cells known in the art, thereby confirming an effect of suppressing fibrosis. Accordingly, the same can be usefully employed in the prevention or treatment of fibrosis.

### BRIEF DESCRIPTION OF DRAWINGS

FIG. 1 shows a conceptual diagram of the antifibrotic effect of directly reprogrammed natural killer (drNK) cells and conditioned medium (CM) obtained by culturing the same.

FIGS. 2A and 2B shows representative types of NK cells exhibiting different CD56 and CD16 expression patterns that were comparatively analyzed to identify natural killer cell types effective for the antifibrotic activity of the present invention. FIG. 2A shows the following NK cells exhibiting different expression patterns of CD56 [Dim (Low), Bright (High), Superbright (Superhigh)] and CD16 CD56 [Dim (Low), Bright (High), Superbright (Superhigh)]: 1. CD56^{dim}CD16^{bright} pNK (CD56^{dim} pNK), the main cell type of PBMC-derived (pNK) NK cells; 2. IL-2/IL-15 cytokine-activated CD56^{bright}CD16^{bright} pNK (CD56^{bright} pNK); 3. NK92, a CD56^{bright}CD16^{dim}-immortalized NK cell line; 4. CD56^{bright}CD16^{dim} iPS-NK differentiated from iPSCs; and 5. CD56^{superbright}CD16^{superbright}drNK cells produced through direct reprogramming of the present invention. FIG. 2B shows results confirming the CD56 and CD16 expression patterns of each NK cell in FIG. 2A by flow cytometry.

FIGS. 3A-3C shows the killing ability of drNK against hepatic fibrogenic human hepatic stellate cells (HSCs). FIG. 3A shows results confirming the increased expression of type 1 collagen and alpha-SMA proteins in activated human hepatic stellate cells (activated HSCs, aHSCs) treated with TGF-β compared to quiescent HSCs (qHSCs) by immunohistochemical staining. FIG. 3B shows results confirming the increased collagen deposition in aHSCs by Picrosirius red staining. FIG. 3C shows results confirming the superior aHSC-killing ability of drNK relative to the control groups (CD56^{dim} pNK, CD56^{bright} pNK, NK92, and iPS-NK).

FIGS. 4A-4C shows the antifibrotic effect of drNK dependent on CD56 and CD16 expression. FIG. 4A shows a schematic diagram of the aHSC cytotoxicity assay using anti-CD56 and anti-CD16 antibodies to analyze the correlation between aHSC cytotoxicity and CD56 and CD16 expression. FIG. 4B shows results confirming that, upon conducting the anti-CD56 antibody blocking assay of FIG. 4A, aHSC cytotoxicity was most significantly suppressed by CD56 blocking in the drNK of the present invention compared to the control groups (CD56^{dim} pNK, CD56^{bright} pNK, NK92, and iPS-NK), thereby confirming that the high antifibrotic activity of drNK is associated with CD56 expression. FIG. 4C shows results confirming that, upon conducting the anti-CD16 antibody blocking assay of FIG. 4A, aHSC cytotoxicity was most significantly suppressed by CD16 blocking in the drNK of the present invention compared to the control groups (CD56^{dim} pNK, CD56^{bright} pNK, NK92, and iPS-NK), thereby confirming that the high antifibrotic activity of drNK is associated with CD16 expression.

FIGS. 5A-5C shows the antifibrotic effect of drNK dependent on NKG2D expression. FIG. 5A shows results confirming that the drNK of the present invention exhibits relatively higher NKG2D expression characteristics compared to control NK cells [peripheral blood (pNK), placenta (UCB-NK), embryonic stem cell-differentiated (ESC-NK), and induced pluripotent stem cell-differentiated (iPS-NK)] (FIG. 5A, left), and that aHSC exhibits relatively higher MICA (NKG2D ligand) expression characteristics compared to control qHSC (FIG. 5A, right). FIG. 5B shows a schematic diagram of the aHSC cytotoxicity assay using an anti-NKG2D antibody to analyze the correlation between aHSC cytotoxicity and NKG2D expression. FIG. 5C shows results confirming that, upon conducting the anti-NKG2D antibody blocking assay of FIG. 5B, the cytotoxicity of drNK, which exhibits relatively higher NKG2D expression, was most significantly suppressed by NKG2D blocking compared to the control groups (CD56^{dim} pNK, CD56^{bright} pNK, NK92, and iPS-NK), thereby confirming that the high antifibrotic activity of drNK is associated with NKG2D expression.

FIGS. 6A-6F shows the antifibrotic effect of drNK dependent on CCR5 expression. FIGS. 6A-6F shows results confirming the relatively higher CCR5 mRNA (FIG. 6A) and protein expression (FIG. 6B) of drNK compared to existing NK cells (pNK and iPS-NK). FIG. 6C shows a schematic diagram of the aHSC cytotoxicity assay using an anti-CCR5 antibody to analyze the correlation between aHSC cytotoxicity and CCR5 expression. FIG. 6D shows results confirming that, upon conducting the anti-CCR5 antibody blocking assay of FIG. 6C, the cytotoxicity of drNK was most significantly suppressed by CCR5 blocking compared to the control groups (pNK, NK92, and iPS-NK), thereby confirming that the high antifibrotic activity of drNK, which exhibits relatively higher CCR5 expression, is associated with CCR5 expression. FIG. 6E shows results confirming high CCL5 expression patterns in the livers of fibrotic mice. FIG. 6F shows results verifying the utility of transplanted drNK as an antifibrotic cell therapy agent, by intravenously injecting into mice pNK and drNK, respectively, followed by extraction of the liver and spleen to examine residual NK cells through flow cytometry with an anti-human CD45 antibody, whereby drNK was confirmed to remain at higher levels in the liver compared to pNK.

FIGS. 7A and 7B shows the superior antifibrotic effect of drNK in a fibrotic liver organoid model. FIG. 7A shows the results of preparing a multicellular liver organoid model from iPSs and confirming the expression of hepatic differentiation characteristic markers. FIG. 7B shows results confirming the superior antifibrotic effect of drNK compared to pNK in an oleic acid (sodium oleate; OA)-mediated fibrotic liver organoid model, through analysis of cells positive for the lipophilic fluorescent dye BODIPY493/503, collagen (anti-COL1A1), and fibrous extracellular matrix staining (Sirius red).

FIGS. 8A-8C shows the inhibitory effect of drNK on body weight and liver weight reduction in a mouse model of nonalcoholic steatohepatitis (NASH) induced by a methionine-choline deficient diet (MCD-diet). FIG. 8A shows a schematic diagram of the animal experiment method for the NASH-induced MCD-diet mouse model. FIG. 8B shows results confirming the superior inhibitory effect of directly reprogrammed natural killer cells on body weight reduction compared to the control groups (pNK and Poly IC) at 9 weeks following administration in FIG. 8A. FIG. 8C shows results confirming the superior inhibitory effect of directly reprogrammed natural killer cells on liver tissue weight reduction compared to the control groups (pNK and Poly IC) at 9 weeks following administration in FIG. 8A.

FIGS. 9A-9D shows the antifibrotic effect of drNK through suppression of liver damage indicators and steatosis in the NASH-induced MCD-diet mouse model. FIG. 9A shows results confirming the superior inhibitory effect of drNK on the increase in liver damage indicators (ALT and AST) compared to the control groups (pNK and Poly IC) at 9 weeks following cell transplantation in FIG. 8A. FIG. 9B shows the results of assessing structural loss in liver tissue by Hematoxylin & Eosin staining, the degree of steatosis (steatosis score) by Oil Red O staining, and the degree of collagen development (fibrosis score) by Masson's Trichrome staining in FIG. 8A. At 9 weeks following cell transplantation, superior recovery of structural loss (FIG. 9B), suppression of steatosis progression (FIGS. 9B and 9C), and suppression of collagen tissue development (FIGS. 9B and 9D) were observed in drNK compared to the control groups (pNK and Poly IC).

FIG. 10 shows the antifibrotic effect of drNK through suppression of fibrosis marker expression in the NASH-induced MCD-diet mouse model. The gene expression of fibrosis markers (α-SMA, TGF-β, TIMP-1, COL1A1, COL3A1) and extracellular matrix degradation genes (MMP-1, MMP-2) was analyzed via RT-qPCR at 9 weeks following cell transplantation in FIG. 8A. The results confirmed that drNK significantly suppressed the expression of fibrosis markers and enhanced the expression of extracellular matrix-degrading MMP-1 and MMP-2 compared to the control groups (pNK and Poly IC).

FIG. 11 shows the antifibrotic effect of drNK through enhancement of anti-inflammatory marker expression and suppression of inflammatory marker expression in the NASH-induced MCD-diet mouse model. The gene expression of anti-inflammatory markers (IL-10, IL-11, IL-4) and inflammatory markers (IL-1β, IL-6, TNF-α) was analyzed via RT-qPCR at 9 weeks following cell transplantation in FIG. 8A. The results confirmed that drNK significantly enhanced the expression of anti-inflammatory markers and suppressed the expression of inflammatory markers compared to the control groups (pNK and Poly IC).

FIGS. 12A-12D shows the inhibitory effects of conditioned medium from drNK on the fibrogenic activity of human hepatic stellate cells. FIG. 12A shows the analysis of the gene expression of fibrosis markers LOXL2, COL1A1, and ACTA2 by RT-qPCR in human hepatic stellate cells cultured in the presence or absence (+/−) of TGF-β or NK-CM. It was confirmed that the gene expression of fibrosis markers LOXL2, COL1A1, and ACTA2 significantly decreased in the group treated with conditioned medium from drNK (CM-drNK) compared to the control group (pNK-CM). FIG. 12B shows results (cell fluorescence staining images and quantification graphs) confirming the superior inhibitory effect of CM-drNK on type 1 collagen and α-SMA protein expression compared to the control group (CM-pNK), by immunohistochemical staining in human hepatic stellate cells cultured in the presence or absence (+/−) of TGF-β or NK-CM. FIG. 12C shows results confirming the superior inhibitory effects of CM-drNK on collagen deposition and fibrosis compared to the control group (pNK-CM) by Sirius red collagen staining in human hepatic stellate cells cultured in the presence or absence (+/−) of TGF-β or NK-CM. FIG. 12D shows results confirming the superior inhibitory effect of CM-drNK on aHSC cell proliferation compared to the control group (pNK-CM).

FIGS. 13A-13D shows the killing ability of drNK against pulmonary fibrogenic human lung fibroblasts (MRCs). FIG. 13A shows the results of confirming by RT-qPCR the increased gene expression of COL1A1 and COL3A1 in human lung fibroblasts activated by TGF-β treatment (activated MRC, aMRC) compared to quiescent MRC (qMRC). FIG. 13B shows results confirming increased collagen deposition in aHSCs by Picrosirius red staining. FIG. 13C shows results confirming increased type 1 collagen and alpha-SMA protein expression in aMRC by immunohistochemical staining. FIG. 13D shows results confirming the superior killing ability of drNK against aMRC compared to control NK92 cells.

FIGS. 14A-14D shows the inhibitory effects of conditioned medium from drNK on the fibrogenic activity of human lung fibroblasts. FIG. 14A shows the analysis of the gene expression of fibrosis markers COL1A1, COL3A1, ACTA2, MYH9, MYH10, MYH14, and MMP2 in human lung fibroblasts (MRC-5) cultured in the presence or absence (+/−) of TGF-β or NK-CM. It was confirmed that the gene expression of COL1A1, COL3A1, ACTA2, MYH9, MYH10, MYH14, and MMP2 significantly decreased in the group treated with conditioned medium from drNK (CM-drNK) compared to the control group (CM-pNK). FIG. 14B shows results confirming the superior inhibitory effect of CM-drNK on type 1 collagen and alpha-SMA protein expression compared to the control group (pNK-CM) by immunohistochemical staining in human lung fibroblasts (MRC-5) cultured in the presence or absence (+/−) of TGF-β or NK-CM. FIG. 14C shows results confirming the superior inhibitory effects of CM-drNK on collagen deposition and fibrosis compared to the control group (pNK-CM) by Sirius red collagen staining in human lung fibroblasts cultured in the presence or absence (+/−) of TGF-β or NK-CM. FIG. 14D shows results confirming the superior inhibitory effect of CM-drNK on aMRC-5 cell proliferation compared to the control group (pNK-CM).

FIGS. 15A-15D shows the killing ability of drNK against cardiac fibrogenic human cardiac fibroblasts (IM-HCF). FIG. 15A shows results confirming the increased expression of fibrosis marker COL1A1, TIMP1, and ACTA2 mRNAs in human cardiac fibroblasts activated by TGF-β treatment (aIM-HCF) compared to quiescent IM-HCF (qIM-HCF) by RT-qPCR. FIG. 15B shows results confirming increased collagen deposition in aIM-HCFs by Picrosirius red staining. FIG. 15C shows results confirming increased type 1 collagen and alpha-SMA protein expression in aIM-HCF by immunohistochemical staining. FIG. 15D shows results confirming the superior killing ability of drNK against aIM-HCF compared to control NK92 cells.

FIGS. 16A-16D shows the inhibitory effects of conditioned medium from drNK on the fibrogenic activity of human cardiac fibroblasts. FIG. 16A shows the analysis of the gene expression of fibrosis markers COL1A1, CILP1, THBS4, VEGF-A, and MMP9 by RT-qPCR in human cardiac fibroblasts (IM-HCF) cultured in the presence or absence (+/−) of TGF-β or NK-CM. It was confirmed that the gene expression of COL1A1, CILP1, THBS4, VEGF-A, and MMP9 significantly decreased in the group treated with conditioned medium from drNK (CM-drNK) compared to the control group (CM-pNK). FIG. 16B shows results confirming the superior inhibitory effects of CM-drNK on type 1 collagen and alpha-SMA protein expression compared to the control group (pNK-CM) by immunohistochemical staining in human cardiac fibroblasts (IM-HCF) cultured in the presence or absence (+/−) of TGF-β or NK-CM. FIG. 16C shows results confirming the superior inhibitory effects of CM-drNK on collagen deposition and fibrosis compared to the control group (pNK-CM) by Picrosirius red collagen staining in human cardiac fibroblasts (IM-HCF) cultured in the presence or absence (+/−) of TGF-β or NK-CM. FIG. 16D shows results confirming the superior inhibitory effect of CM-drNK on aIM-HCF cell proliferation compared to the control group (pNK-CM).

FIGS. 17A-17D shows the killing ability of drNK against dermal fibrogenic human skin fibroblasts. FIG. 17A shows results confirming the increased gene expression of fibrosis markers COL1A1, COL3A1, and ACTA2 in human skin fibroblasts activated by TGF-β treatment (aHDF) compared to control quiescent HDF (qHDF) by RT-qPCR. FIG. 17B shows results confirming increased collagen deposition in aHDFs by Picrosirius red staining. FIG. 17C shows results confirming increased type 1 collagen and alpha-SMA protein expression in aHDF by immunohistochemical staining. FIG. 17D shows results confirming the superior killing ability of drNK against aHDF compared to control NK92 cells.

FIGS. 18A-18D shows the inhibitory effects of conditioned medium from drNK on the fibrogenic activity of human skin fibroblasts (HDF). FIG. 18A shows the analysis of the gene expression of fibrosis markers COL1A1, COL3A1, and ACTA2 by RT-qPCR in human skin fibroblasts (HDF) cultured in the presence or absence (+/−) of TGF-β or NK-CM. It was confirmed that the gene expression of fibrosis markers COL1A1, COL3A1, and ACTA2 significantly decreased in the group treated with conditioned medium from drNK (CM-drNK) compared to the control group (CM-pNK). FIG. 18B shows results confirming the superior inhibitory effects of CM-drNK on type 1 collagen and alpha-SMA protein expression compared to the control group (pNK-CM) by immunohistochemical staining in aHDF. FIG. 18C shows results confirming the superior inhibitory effects of CM-drNK on collagen deposition and fibrosis compared to the control group (pNK-CM) by Picrosirius red collagen staining in human skin fibroblasts (HDF) cultured in the presence or absence (+/−) of TGF-β or NK-CM. FIG. 18D shows results confirming the superior inhibitory effect of CM-drNK on aHDF cell proliferation compared to the control group (pNK-CM).

FIGS. 19A-19D shows the killing ability of drNK against renal fibrogenic human renal tubular epithelial cells. FIG. 19A shows results confirming the increased gene expression of fibrosis markers COL1A1, COL3A1, and ACTA2 in human renal tubular epithelial cells activated by TGF-β treatment (aHK-2) compared to control quiescent HK-2 (qHK-2) by RT-qPCR. FIG. 19B shows results confirming increased collagen deposition in aHK-2 by Picrosirius red staining. FIG. 19C shows results confirming increased type 1 collagen and alpha-SMA protein expression in aHK-2 by immunohistochemical staining. FIG. 19D shows results confirming the superior killing ability of drNK against aHK-2 compared to control NK92 cells.

FIGS. 20A-20D shows the inhibitory effects of conditioned medium from drNK on the fibrogenic activity of human renal tubular epithelial cells (aHK-2). FIG. 20A shows the analysis of the gene expression of fibrosis markers COL3A1, ACTA2, and PAI-1 by RT-qPCR in HK-2 cultured in the presence or absence (+/−) of TGF-β or NK-CM. It was confirmed that the gene expression of fibrosis markers COL3A1, ACTA2, and PAI-1 significantly decreased in the group treated with conditioned medium from drNK (CM-drNK) compared to the control group (CM-pNK). FIG. 20B shows results confirming the superior inhibitory effects of CM-drNK on type 1 collagen and alpha-SMA protein expression compared to the control group (pNK-CM) by immunohistochemical staining in aHK-2. FIG. 20C shows results confirming the superior inhibitory effects of CM-drNK on collagen deposition and fibrosis compared to the control group (pNK-CM) by Picrosirius red collagen staining in HK-2 cultured in the presence or absence (+/−) of TGF-β or NK-CM. FIG. 20D shows results confirming the superior inhibitory effect of CM-drNK on aHK-2 cell proliferation compared to the control group (pNK-CM).

FIG. 21 shows the quantitative comparative analysis of cytokine gene expression levels in NK-92, iPS-NK, and drNK by qPCR. Ten cytokines (CCL5, IFN-γ, CXCL11, CXCL12, GDNF, VEGF, XCL1, IL16, LIF, and LTB) that are highly expressed in drNK compared to controls NK-92 and iPS-NK were identified.

FIGS. 22A-22C shows the comparative analysis of the profiles of endogenous cytokines in the conditioned media from CD56^{dim} pNK and drNK using a human proteome cytokine array. FIG. 22A is a graph showing the 56 secretory proteins identified in the conditioned medium from drNK cells. FIG. 22B is a graph showing the 28 secretory proteins that are increased in the drNK-conditioned medium compared to the CD56^{dim} pNK cell-conditioned medium. FIG. 22C shows the proteins DPP4, M-CSF, and BDNF identified specifically in the drNK cell-conditioned medium.

FIG. 23 shows a comparison of the proportions of CD56, CD16, NKG2D, and CCR5 positive cells between pNK and drNK. It is demonstrated that drNK exhibits a significantly increased proportion of CD16, NKG2D, and CCR5 positive cells compared to pNK, and in particular, that drNK is a highly homogeneous cell population simultaneously expressing CD56, CD16, NKG2D, and CCR5. FIG. 24 shows a comparison of the mean fluorescence intensity (MFI) of CD56, CD16, NKG2D, and CCR5 between pNK and drNK. drNK exhibits significantly increased expression intensity of CD56, CD16, and CCR5 compared to pNK, indicating that drNK is a cell population having a quantitatively distinct “superbright” phenotype rather than simply positive cells.

FIGS. 25A-25D shows the mechanism by which ligands corresponding to NK cell receptors are increased in activated hepatic stellate cells (aHSC), and by which drNK recognizes and eliminates fibrotic cells through CCR5, CCR9, and CX3CR1-related axes. FIGS. 25A and 25B show the increased expression of CCL5, CCL25, and CX3CL1 in aHSC, and FIGS. 25C and 25D show the reduction in killing ability of drNK against aHSC upon blocking of ligands or receptors.

FIG. 26 shows a comparison of the expression of CCL3, CCL4, CCL5, and MICA in a three-dimensional fibrotic organoid model. Under fibrogenic conditions, the expression of the above ligands is increased compared to the control group, demonstrating that the multi-ligand-receptor-based antifibrotic action of drNK is also applicable in tissue-like environments.

### DETAILED DESCRIPTION OF PREFERRED EMBODIMENTS

The present invention will be described in detail as follows. Meanwhile, each description and embodiment described herein may be applied to other descriptions and embodiments, respectively. That is, all combinations of various elements described herein fall within the scope of the present invention. Further, the scope of the present invention is not limited by the specific description described below.

One aspect of the present invention for achieving the above objects provides a pharmaceutical composition for preventing or treating fibrosis, comprising directly reprogrammed natural killer (drNK) cells expressing one or more selected from the group consisting of CD56^{superbright}, CD16^{superbright}, and a combination thereof.

The inventors of the present invention have for the first time established that when the composition is applied to a fibrotic cell model, collagen production, activity, and viability are significantly reduced, thereby demonstrating excellent efficacy in suppressing fibrosis.

As used herein, the term “superbright” does not refer simply to whether expression has occurred, but rather refers to a state of high expression in which mean fluorescence intensity (MFI) is increased compared to that of control natural killer cells measured under identical conditions. “Superbright” may be a state in which the MFI of at least one of CD56 or CD16 is increased by at least 2-fold, specifically at least 5-fold, compared to control natural killer cells.

In addition, in one example, the drNK cells may further express one or more receptors selected from the group consisting of NKG2D, CCR5, CCR9, and CX3CR1 in addition to CD56 and CD16, and the receptors may mediate migration, adhesion, and cytotoxicity toward target cells through interaction with ligands expressed on fibrotic cells.

In another example, the drNK cells may be a homogeneous cell population in which the proportion of cells simultaneously expressing CD56, CD16, NKG2D, and CCR5 is 80% or more of the total cells.

In particular, the directly reprogrammed natural killer (drNK) cells of the present invention exhibit higher expression of NK cell activation receptors, such as CD69, NKG2D, DNAM-1, and NKp46, compared to control NK cells. Further, ten cytokine/chemokine genes overexpressed in drNK were observed, as well as three proteins identified specifically and exclusively in drNK.

Moreover, it was confirmed that directly reprogrammed natural killer (drNK) cells of the present invention expressing one or more selected from the group consisting of CD56^{superbright}, CD16^{superbright}, and a combination thereof exhibit a superior direct killing ability compared to control natural killer (NK) cells known in the art when treated against hepatic stellate cells (hepatic fibrosis cell model), alveolar epithelial cells (pulmonary fibrosis cell model), cardiac cells (cardiac fibrosis cell model), skin cells (dermal fibrosis cell model), and renal cells (renal fibrosis cell model), which are fibrosis-causing cells associated with the induction of fibrotic responses in each respective tissue. Furthermore, when conditioned medium (CM) from the drNK cells was applied to the fibrosis cell models above, collagen production, activity, and viability thereof were significantly reduced compared to that from control natural killer (NK) cells known in the art, thereby confirming an effect of suppressing fibrosis.

This suggests that the composition of the present invention is useful in the prevention or treatment of various fibrotic diseases when treated with the drNK of the present invention compared to NK cells known in the art.

The drNK cells of the present invention exhibit cytotoxicity against fibrotic cells through NKG2D or CCR5 receptors, and are characterized in that their cytotoxicity is reduced when these receptors are blocked. In addition, the drNK cells of the present invention constitute a homogeneous cell population exhibiting anti-fibrosis-related receptors, including NKG2D and CCR5 in addition to CD56 and CD16, at a high proportion and with high expression intensity. Accordingly, unlike existing pNK or activated NK cells, the drNK cells of the present invention can be used as a quantitatively identifiable raw material for a cell therapy agent from the perspectives of preparation and quality control.

Furthermore, the drNK cells of the present invention recognize ligands such as CCL5, CCL25, CX3CL1, and MICA that are increased in fibrotic cells, and exhibit the functions of migrating to, adhering to, and eliminating fibrotic cells through multiple receptor axes including CCR5, CCR9, CX3CR1, and NKG2D. Accordingly, the drNK cells of the present invention can perform selective antifibrotic action based on molecular changes in fibrotic cells, rather than non-specific effects resulting from a simple increase in NK cell activity.

Specifically, the directly reprogrammed natural killer cells of the present invention may be prepared by direct reprogramming of isolated cells.

In one specific embodiment, the directly reprogrammed natural killer cells may be prepared by a method comprising: (a) introducing a reprogramming factor into isolated cells; and (b) culturing the same in a medium comprising cytokines, growth factors, a glycogen synthase kinase 3β (GSK3β) inhibitor, and an aryl hydrocarbon receptor (AHR) inhibitor to produce natural killer cells; but are not limited thereto.

In the present invention, step (a) introduces one or more reprogramming factors into isolated cells.

As used herein, the term “isolated cells” is not particularly limited, but may specifically refer to cells whose lineage has already been specified, such as germ cells, somatic cells, or progenitor cells. For example, the cells may be derived from humans, but cells derived from various entities also fall within the scope of the present invention.

The isolated cells may be derived from humans, but are not limited thereto, and cells derived from various entities may also fall within the scope of the present invention. In addition, the isolated cells of the present invention may include both in vivo and ex vivo cells.

Furthermore, the isolated cells specifically may be somatic cells excluding natural killer cells.

As used herein, the term “reprogramming factor” refers to a gene (or a polynucleotide encoding the same) or a protein that can be introduced into a cell to induce reprogramming. The reprogramming factor may vary depending on the target cell into which reprogramming is to be induced, as well as the type of isolated cells in which reprogramming is induced. For example, when preparing natural killer cells, the reprogramming factor may include one or more factors selected from the group consisting of Lin28, Ascl1, Pitx3, Nurr1, Lmx1a, Nanog, Oct3, Oct4, Sox2, Klf4, and Myc, and may include all other factors known in the art as being capable of preparing natural killer cells. In addition, direct reprogramming into natural killer cells can be induced using the reprogramming factor. In direct reprogramming methodology, there is a method using reprogramming genetic factors, and the vector of the present invention may be used for such purpose. Accordingly, those skilled in the art can select appropriate factors depending on the target cell type and the type of cells before reprogramming, and the type thereof is not particularly limited as all factors fall within the scope of the present invention as long as they are within the scope of knowledge in the art. Since reprogramming using reprogramming genetic factors induces conversion into the target cell by regulating the overall gene expression pattern of the cell, the initial cells can be reprogrammed into target cells having the gene expression pattern of the target cell type by introducing the reprogramming genetic factor into the cells and culturing the cells for a certain period of time. In the present invention, the term “reprogramming factor” may be used interchangeably with “direct reprogramming-inducing factor”, “direct cell conversion-inducing factor”, and “cell conversion-inducing factor”.

In the present invention, “introducing reprogramming factors” may refer to a method of increasing the expression level of reprogramming factors present within a cell, particularly the Oct4, Sox2, Klf4, and c-Myc genes; or a method of increasing the expression level of reprogramming factors within a cell through expression vectors, genetic modification, introduction of exogenous expression genes, treatment with substances having expression-inducing effects, etc., but is not limited thereto, as long as the expression level of the reprogramming factors is increased. In particular, introducing reprogramming factors may refer to a method of inducing the expression of reprogramming factors at a desired time under desired conditions.

Specifically, the method of introducing the reprogramming factors into cells of (a) may be used without limitation in providing nucleic acid molecules (DNA or RNA) or proteins to cells conventionally used in the art. For example, a method of administering reprogramming factors to the cell culture medium, a method of directly injecting reprogramming factors into cells, or a method of transforming cells using an expression vector carrying the genes for reprogramming factors may be used.

The method of directly injecting reprogramming factors into cells may be selected from any method known in the art, and may be appropriately selected and applied from, without limitation, microinjection, electroporation, particle bombardment, direct intramuscular injection, and methods utilizing insulators and transposons.

As used herein, the term “expression vector” refers to a vector capable of expressing a target protein in a suitable host cell, and refers to a genetic construct comprising essential regulatory elements operably linked to a gene insert to enable the expression thereof.

The expression vector of the present invention comprises, in addition to expression regulatory elements such as a promoter, operator, initiation codon, termination codon, polyadenylation signal, and enhancer, a signal sequence or leader sequence for membrane targeting or secretion, and may be constructed in various ways depending on the intended purpose. The promoter of the expression vector may be constitutive or inducible. In addition, the expression vector comprises a selection marker for selecting host cells containing the vector, and in the case of a replicable expression vector, comprises an origin of replication. The expression vector may self-replicate or integrate into host DNA.

The expression vector may include viral vectors, episomal vectors, plasmid vectors, cosmid vectors, etc., but is not limited thereto

Specifically, the viral vector may include vectors derived from lentiviruses; retroviruses such as human immunodeficiency virus (HIV), murine leukemia virus (MLV), avian sarcoma/leukosis virus (ASLV), spleen necrosis virus (SNV), Rous sarcoma virus (RSV), and mouse mammary tumor virus (MMTV); adenoviruses, adeno-associated viruses, herpes simplex viruses, etc. In addition, the vector may more specifically be an RNA-based viral vector, but is not limited thereto.

In addition, the episomal vector of the present invention is a non-viral, non-integrating vector known to express genes comprised in the vector without insertion into a chromosome. For the purpose of the present invention, cells comprising an episomal vector include both cases where the episomal vector is inserted into the genome, and where it exists within the cell without being inserted into the genome. Furthermore, the episomal vector may comprise one or more reprogramming factors.

As used herein, the term “operably linked” refers to the functional linkage between a nucleic acid expression regulatory sequence and a nucleic acid sequence encoding a target protein so as to perform a general function. Operable linkage with a recombinant vector can be constructed using genetic recombination techniques well known in the art, and site-specific DNA cleavage and ligation employ enzymes, etc. commonly known in the art.

Next, step (b) directly reprograms the cells of (a) into natural killer (NK) cells by culturing in i) a first medium comprising cytokines, growth factors, and a GSK3β inhibitor, and ii) a second medium comprising cytokines and growth factors.

As used herein, the term “cytokine” refers to various, relatively small proteins produced by cells and used in cell signaling, which can affect other cells including those that produce them. They are generally associated with immune responses to inflammation or infection, but are not limited thereto. Specifically, the cytokine may be IL-2, IL-3, IL-5, IL-6, IL-7, IL-11, IL-15, BMP4, Acivin A, Notch ligand, G-CSF, or SDF-1, but is not limited thereto.

As used herein, the term “growth factor” refers to a polypeptide that promotes the division, growth, and differentiation of various cells, and includes epidermal growth factor (EGF), platelet-derived growth factor-AA (PDGF-AA), insulin-like growth factor-1 (IGF-1), transforming growth factor-β (TGF-β), and fibroblast growth factor (FGF), but is not limited thereto.

For the purpose of the present invention, the cytokines and growth factors are comprised in the medium for directly reprogramming isolated cells into lineage-converted cells, and the types of cytokines and growth factors are not limited as long as they are used for direct reprogramming.

As used herein, the term “GSK3β inhibitor” refers to a substance that suppresses or inhibits the activity of GSK3β, which is one of the two isoforms of glycogen synthase kinase-3 (GSK3). The GSK3β inhibitor may include lithium, SB216763, CHIR-98014, TWS119, AR-A014418, etc., and may specifically be CHIR99021, but is not limited thereto.

As used herein, the term “culturing” refers to growing cells under appropriately controlled environmental conditions, and the culturing process of the present invention may be performed according to suitable media and culture conditions known in the art. Such culturing processes can be readily adjusted and used by those skilled in the art depending on the selected cells.

In particular, since step (b) is a process of culturing cells with introduced reprogramming factors in the first medium and the second medium, the compositions of the first medium and the second medium for culturing the cells are suitable for directly reprogramming the cells with introduced reprogramming factors into natural killer cells; specifically, the first medium may comprise cytokines, growth factors, and a GSK3β inhibitor, and the second medium may comprise cytokines and growth factors, but are not limited thereto.

As used herein, the term “medium” refers to a known medium used in cell culture, and encompasses all known cell culture media or modified versions thereof.

The term “StemRegenin I” as used in the second medium is an aryl hydrocarbon receptor inhibitor, and refers to 4-(2-((2-benzo[b]thiophen-3-yl)-9-isopropyl-9H-purin-6-yl)amino)ethyl)phenol hydrochloride. Stemregenin I may be further comprised with IL-7 and IL-15 in addition to the cytokines, growth factors, and GSK3β inhibitor of the first medium, and may be used to increase the efficiency of direct reprogramming.

Specifically, the first medium and the second medium increase the efficiency of direct reprogramming to promote natural killer cell production. The first medium comprises cytokines, growth factors, and a GSK3β inhibitor, and may specifically comprise FBS, CHIR99021, IL-3, IL-6, SCF, FLT3L, and TPO, but is not limited thereto. In addition, the second medium comprises cytokines, growth factors, and StemRegenin I, and may specifically comprise FBS, IL-2, IL-7, IL-15, SCF, FLT3L, and StemRegenin I, but is not limited thereto as long as the medium composition increases the efficiency of direct reprogramming.

As used herein, the term “reprogramming” refers to a method of converting a specific cell into a target cell by regulating the global gene expression pattern, etc. of the specific cell. In other words, reprogramming in the present invention refers to a method of artificially manipulating the fate of a cell to convert it into a cell with entirely different properties. For the purpose of the present invention, the reprogramming may be performed by introducing into the cell a vector comprising a foreign gene or DNA. For example, reprogramming may include dedifferentiation, direct reprogramming (or direct conversion), or direct trans-differentiation of cells, but is not limited thereto.

As used herein, the term “direct reprogramming” differs from the technique of producing induced pluripotent stem cells having pluripotency through a reprogramming process, and refers to a technique that directly induces conversion to the desired target cell through reprogramming culture. In order to produce the target natural killer cells using existing induced pluripotent stem cell reprogramming technology, it is required to first produce induced pluripotent stem cells from isolated somatic cells and then differentiate the same to produce hematopoietic stem (progenitor) cells as intermediates. Subsequently, the final target natural killer cells should be differentiated and produced from hematopoietic stem (progenitor) cells, which were induced to differentiate from induced pluripotent stem cells. Since it is required to sequentially undergo such a complex production and culturing process, there are disadvantages of low production efficiency and high consumption of time and cost. Furthermore, since the cells are produced via pluripotent stem cells, the presence of residual undifferentiated cells and safety assurance have become important issues that must be addressed. However, the present invention is expected to provide an alternative technique capable of overcoming the above problems such as production time, cost, efficiency, and safety by directly producing the target natural killer cells from initial cells through direct reprogramming technology. For the purpose of the present invention, direct reprogramming may be used interchangeably with direct dedifferentiation, direct differentiation, direct conversion, direct trans-differentiation, trans-differentiation, etc. In the present invention, direct reprogramming may in particular refer to direct dedifferentiation or trans-differentiation into natural killer cells.

As used herein, the term “differentiated cell” refers to a cell that has become specialized in structure or function. That is, it refers to a state in which cells, tissues, etc. of an organism have changed into suitable forms and functions to perform their respective assigned roles. For example, ectoderm, mesoderm, and endoderm cells derived from pluripotent stem cells such as embryonic stem cells are differentiated cells, and in a narrower sense, red blood cells, white blood cells, and platelets derived from hematopoietic stem cells are also differentiated cells.

As used herein, the terms “intermediate cell” or “natural killer progenitor cell” refer to a cell at a stage prior to differentiation into natural killer cells, and may be a lymphoid stem cell, but are not limited thereto as long as there is a possibility of differentiating into natural killer cells.

As used herein, the term “natural killer (NK) cell” refers to a key innate immune cell that immediately recognizes and eliminates infections caused by viruses, germs (bacteria), fungi, and parasites, as well as abnormal autologous cells. Unlike T cells, which recognize target cells by expressing antigen-specific receptors, NK cells recognize, without antigen specificity or human leukocyte antigen (HLA) matching, abnormal changes in target cells (particularly cancer cells and infected cells) through the balance of inhibitory or activating receptors such as killer immunoglobulin receptors (KIR), natural cytotoxicity receptors (NCR), DNAX accessory molecule-1 (DNAM-1), and NK group 2 member D (NKG2D), the loss of surface major histocompatibility complex (MHC) class I antigens, etc., and exhibit contact-dependent cytotoxicity through various mechanisms. Unlike T cells, which can induce graft-versus-host disease (GVHD) against non-self allogeneic cells with mismatched human leukocyte antigens (HLA), allogeneic NK cells have been shown to exhibit virtually no adverse effects of graft-versus-host disease and instead to demonstrate strong therapeutic effects.

For the purpose of the present invention, natural killer cells refer to those obtained through direct reprogramming, and may be used interchangeably with directly reprogrammed natural killer (drNK) cells.

Another specific aspect of the present invention provides directly reprogrammed natural killer (drNK) cells prepared by the above method. The directly reprogrammed natural killer cells prepared by the method of the present invention may express at least one selected from the group consisting of CD56^{superbright}, CD16^{superbright}, and a combination thereof, but are not limited thereto.

In another specific embodiment, the directly reprogrammed natural killer (drNK) cells may overexpress, compared to a control group, one or more genes selected from the group consisting of CCL5, IFN-γ, CXCL11, CXCL12, GDNF, VEGF, XCL1, IL16, LIF, and LTB, but are not limited thereto.

In yet another specific embodiment, the directly reprogrammed natural killer (drNK) cells may express one or more proteins selected from the group consisting of DPP4, M-CSF, and BDNF, but are not limited thereto.

In addition, the composition specifically may further comprise NKG2D or CCR5, which are substances secreted from directly reprogrammed natural killer (drNK) cells.

In one specific embodiment of the present invention, in order to compare the drNK of the present invention with existing NK, fresh primary NK (pNK) cells isolated from PBMC, pNK cells activated with IL-2 and IL-15 (ApNK cells), or NK cell lines (NK92, ATCC) were used as control groups. The NK cell phenotypes were distinguished into the following major cell groups according to the expression of CD56 and CD16 markers: drNK: CD56^{superbright}CD16^{superbright}, NK92: CD56^{bright}CD16^{dim}, pNK(CD56^{dim}pNK): CD56^{dim}CD16^{bright}, ApNK(CD56^{bright}pNK): CD56^{bright}CD16^{bright}.

In another specific embodiment of the present invention, as a result of comparing the gene expression of cytokines/chemokines expressed in the drNK of the present invention with that of control NK, ten overexpressed genes (CCL5, IFN-γ, CXCL11, CXCL12, GDNF, VEGF, XCL1, IL16, LIF, and LTB) were observed, and 3 proteins (DPP4, M-CSF, and BDNF) specifically observed in drNK were identified.

As used herein, the term “fibrosis” refers to the development of excessive fibrous connective tissue within an organ or tissue. Fibrosis is characterized by the accumulation and alteration of the extracellular matrix (ECM). Despite having distinct etiological and clinical differences, most chronic fibrotic disorders share in common a consistent stimulus that sustains the production of growth factors, proteolytic enzymes, angiogenic factors, and fibrogenic cytokines, which together stimulate the deposition of connective tissue elements, particularly collagen and proteoglycans, thereby continuously remodeling and destroying normal tissue architecture.

In the present invention, the fibrotic disease may specifically develop from one or more selected from the group consisting of lung, kidney, liver, heart, brain, blood vessels, joints, intestine, skin, soft tissue, bone marrow, penis, peritoneum, muscle, spine, testis, ovary, breast, thyroid, tympanic membrane, pancreas, gallbladder, bladder, prostate, esophagus, stomach, uterus, spleen, lymph nodes, salivary glands, and nervous system, but is not limited thereto.

As used herein, the term “prevention” refers to all actions that suppress or delay the occurrence of fibrosis using a pharmaceutical composition for the prevention or treatment of fibrosis comprising directly reprogrammed natural killer cells expressing at least one selected from the group consisting of CD56^{superbright}, CD16^{superbright}, and a combination thereof.

As used herein, the term “treatment” refers to all actions that ameliorate or cure the symptoms of fibrosis using a pharmaceutical composition for the prevention or treatment of fibrosis comprising directly reprogrammed natural killer cells expressing at least one selected from the group consisting of CD56^{superbright}, CD16^{superbright}, and a combination thereof.

As used herein, the term “pharmaceutical composition” may comprise a pharmaceutically acceptable carrier, and may be formulated into oral dosage forms such as powders, granules, tablets, capsules, suspensions, emulsions, syrups, and aerosols, external preparations, suppositories, and sterile injectable solutions, according to conventional methods.

The pharmaceutically acceptable carrier may include those commonly used in the art, such as lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil, but is not limited thereto. Furthermore, the pharmaceutical composition of the present invention may comprise diluents or excipients such as fillers, extenders, binders, wetting agents, disintegrants, and surfactants, as well as other pharmaceutically acceptable additives.

When the pharmaceutical composition of the present invention is formulated as an oral solid preparation, it may include tablets, pills, powders, granules, capsules, etc., and such solid preparations may comprise at least one excipient, for example, starch, calcium carbonate, sucrose, lactose, gelatin, etc., and may further comprise lubricants such as magnesium stearate and talc, but are not limited thereto.

When the pharmaceutical composition of the present invention is formulated as an oral liquid preparation, it may include suspensions, solutions, emulsions, syrups, etc., and may further comprise diluents such as water and liquid paraffin, as well as wetting agents, sweeteners, flavoring agents, and preservatives, etc., but is not limited thereto.

When the pharmaceutical composition of the present invention is formulated as a parenteral preparation, it may include sterile aqueous solutions, non-aqueous solvents, suspensions, emulsions, lyophilized preparations, and suppositories, and the non-aqueous solvents and suspensions may include propylene glycol, polyethylene glycol, vegetable oils such as olive oil, injectable esters such as ethyl oleate, etc., but are not limited thereto. As a base for suppository, witepsol, macrogol, Tween 61, cacao butter, laurin butter, glycerogelatin, etc. may be used, but are not limited thereto.

In the present invention, the dosage of the pharmaceutical composition may vary depending on the patient's condition, body weight, age, severity of disease, drug formulation, route of administration, and period of administration, but may be appropriately selected by those skilled in the art.

The pharmaceutical composition of the present invention may be administered to mammals such as rats, mice, livestock, and humans via various routes, for example, by oral administration or by intraperitoneal, intravenous, intramuscular, subcutaneous, intrauterine, intrathecal, or intracerebrovascular injection.

In one specific embodiment of the present invention, upon checking the antifibrotic effect of the drNK of the present invention in a NASH-induced MCD-diet mouse model, it was confirmed that, compared to known NK in the art, body weight increased normally, the reduction in liver weight was suppressed, and the efficacy in ameliorating liver damage indicators and steatosis was superior. Furthermore, it was confirmed that, compared to known NK in the art, the expression of hepatic fibrosis markers was suppressed, the expression of inflammatory markers was suppressed, and the expression of anti-inflammatory markers was enhanced. Through these results, it can be understood that the composition comprising the drNK of the present invention is effective in the prevention or treatment of hepatic fibrosis.

In another specific embodiment of the present invention, upon checking the antifibrotic effect of the drNK of the present invention in human pulmonary, cardiac, dermal, or renal fibrosis cell models, it was confirmed that, compared to known NK in the art, the direct killing ability was superior and the expression of fibrosis markers was suppressed. Through these results, it can be understood that the composition comprising the drNK of the present invention is effective in the prevention or treatment of pulmonary, cardiac, dermal, or renal fibrosis.

These results suggest that the directly reprogrammed natural killer (drNK) cells of the present invention are highly effective in the treatment of fibrotic diseases.

Yet another aspect of the present invention for achieving the above objects provides a cell therapy agent for preventing or treating fibrosis, comprising directly reprogrammed natural killer (drNK) cells expressing one or more selected from the group consisting of CD56^{superbright}, CD16^{superbright}, and a combination thereof.

Specifically, the cell therapy agent for preventing or treating fibrosis of the present invention comprises directly reprogrammed natural killer cells (drNK), wherein the drNK cells express at least one selected from the group consisting of CD56^{superbright}, CD16^{superbright}, and a combination thereof, wherein superbright means that the mean fluorescence intensity (MFI) of CD56 or CD16 is at least 2-fold greater compared to that of control natural killer cells measured under the same conditions, and wherein the cell therapy agent is provided as a cryopreserved formulation, an injectable formulation, or a formulation for intravenous administration.

The terms used herein are as described above.

As used herein, the term “cell therapy agent” refers to cells and tissues prepared from an entity through isolation, culture, and special manipulation, used as a pharmaceutical product for therapeutic, diagnostic, and preventive purposes (as regulated by the U.S. FDA); and refers to a pharmaceutical product used for therapeutic, diagnostic, and preventive purposes in order to restore the function of cells or tissues, through a series of actions such as ex vivo proliferation and selection of living autologous, allogeneic, or xenogeneic cells, or otherwise modifying the biological properties of cells.

The cell therapy agent composition may have efficacy in preventing or treating fibrosis by comprising the directly reprogrammed natural killer cells prepared according to the method of the present invention.

The cell therapy agent composition may comprise the directly reprogrammed natural killer cells at 1.0×10 cells/ml to 1.0×10¹⁰ cells/ml, specifically 1.0×10⁶ cells/ml to 1.0×10⁹ cells/ml, based on the total weight of the composition, but is not limited thereto.

The cell therapy agent composition may be formulated into a pharmaceutical preparation of a unit dosage form suitable for administration into the patient's body and administered according to conventional methods in the pharmaceutical field, wherein the preparation comprises an effective dosage for single or multiple administrations. Formulations suitable for such purposes may preferably be parenteral preparations such as injections in the form of ampoules for injection, infusion preparations such as infusion bags, and sprays such as aerosol preparations. The ampoules for injection may be prepared by mixing with an injection solution immediately prior to use, and physiological saline solution, glucose solution, mannitol solution, Ringer's solution, etc. may be used as an injection solution. In addition, infusion bags made of polyvinyl chloride or polyethylene may be used, and examples include infusion bags manufactured by Baxter, Becton Dickinson, Medcep, National Hospital Products, or Terumo.

The pharmaceutical preparation may further comprise, in addition to the active ingredient, one or more pharmaceutically acceptable conventional inert carriers, for example, preservatives, analgesics, solubilizers, or stabilizers in the case of injections, and bases, excipients, lubricants, or preservatives in the case of preparations for topical administration.

The cell therapy agent composition of the present invention or pharmaceutical preparation thereof thus produced may be administered together with or in the form of a mixture with other cells used in fibrosis treatment using administration methods commonly employed in the art, and may specifically be directly engrafted or transplanted to the diseased site of a patient in need of treatment, or directly transplanted or injected into the abdominal cavity, but is not limited thereto. In addition, it is possible to perform the administration by both non-surgical administration using a catheter, and surgical administration method such as injection or transplantation following incision of the diseased site. Furthermore, transplantation by parenteral route according to conventional methods is also possible, for example intravascular injection, in addition to direct administration to the lesion.

The cell therapy agent composition may be administered at a dosage of 0.0001 mg/kg to 1,000 mg/kg per day, specifically 0.01 mg/kg to 100 mg/kg per day, and the administration may be performed once daily or in divided doses several times a day. However, it should be understood that the actual dosage of the active ingredient must be determined in light of various relevant factors, such as the disease to be treated, the severity of the disease, the route of administration, and the patient's body weight, age, and sex. Accordingly, the above dosage does not in any way limit the scope of the present invention.

One aspect of the present invention for achieving the above objects provides a method for preventing or treating fibrosis, comprising administering to a subject suspected of having fibrosis, excluding humans, a pharmaceutical composition for preventing or treating fibrosis, comprising directly reprogrammed natural killer (drNK) cells expressing one or more selected from the group consisting of CD56^{superbright}, CD16^{superbright}, and a combination thereof.

The terms used herein are as described above.

The term “administration” refers to introducing the composition of the present invention into an entity by any appropriate method, and the route of administration of the composition may be via any general route capable of reaching the target tissue. Administration may be performed by intraperitoneal, intravenous, intramuscular, subcutaneous, intradermal, oral, topical, or intranasal routes, but is not limited thereto.

The term “entity” refers to all animals, excluding humans, that have developed or may develop fibrosis, including monkeys, cattle, horses, sheep, pigs, chickens, turkeys, quails, cats, dogs, mice, rats, rabbits, and guinea pigs. The type of entity is not limited, as long as the disease can be effectively prevented or treated by administration of the pharmaceutical composition of the present invention to the entity.

### Mode for Carrying out the Invention

Hereinafter, the present invention will be described in more detail by way of Examples. However, these Examples are given for illustrative purposes only, and the scope of the present invention is not to be construed as being limited to these examples.

### Example 1. Schematic Diagram of the Antifibrotic Effect of Directly Reprogrammed Natural Killer (drNK) Cells

Comparative analysis of the antifibrotic effects of directly reprogrammed natural killer cells prepared through reprogramming culture from isolated somatic cells and conditioned medium therefrom of the present invention with existing NK cells and conditioned medium therefrom using human tissue-specific fibrosis cell models (1. hepatic stellate cell-based hepatic fibrosis cell model, 2. alveolar epithelial cell-based pulmonary fibrosis cell model, 3. cardiac cell-based cardiac fibrosis cell model, 4. skin fibroblast-based dermal fibrosis cell model, and 5. renal tubular epithelial cell-based renal fibrosis cell model) confirmed the superior antifibrotic effect of the specialized directly reprogrammed natural killer cells of the present invention.

### Example 2. Cell Phenotypic Characteristics of the Characterized Directly Reprogrammed Natural Killer Cells of the Present Invention

To obtain drNK cells, the reprogramming factor OSKM was introduced by transfection into PBMC isolated from blood by Ficoll density gradient, after which the PBMC cells and polybrene (4 μg/ml) were co-cultured together for 1 day. The following day, 3×10⁵ of the transfected cells were cultured in a 48-well culture dish for an additional 5 days in culture medium RIM (StemSpan SFEM II comprising 10% FBS, 1% Penicillin/Streptomycin, 5 μM CHIR99021, 20 ng/ml Human IL-3, 20 ng/ml Human IL-6, 20 ng/ml Human SCF, 20 ng/ml Human FLT3L, and 20 ng/ml Human TPO). The cells were cultured for 18 to 40 days in culture medium RMM (StemSpan SFEM II containing 10% FBS, 1% Penicillin-Streptomycin, 200 IU/ml Human IL-2, 20 ng/ml Human IL-7, 20 ng/ml Human IL-15, 20 ng/ml Human SCF, 20 ng/ml Human FLT3L, and 2 μM StemRegenin I). To examine whether drNK cells were prepared through the direct reprogramming above, the cells were stained with anti-CD56-APC (BioLegend) and anti-CD16-PE (BioLegend) antibodies as NK cell markers, and the NK cell (CD56⁺ and CD16⁺) population was subsequently analyzed by flow cytometry.

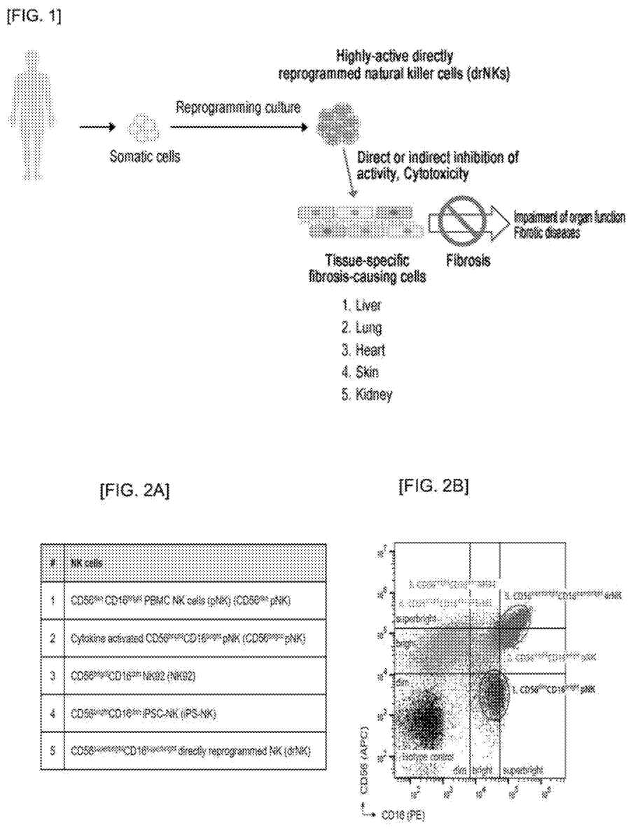

As control cells, fresh primary NK (pNK; CD56^{dim} pNK) cells isolated from PBMC, pNK cells activated with 200 IU/ml Human IL-2 and 20 ng/ml Human IL-15 for 4 to 14 days (cytokine activated pNK; CD56^{bright} pNK), NK cell lines (NK92, ATCC), and iPS-NK cells were prepared as follows: to produce iPS-NK cells, induced pluripotent stem cells (iPS) were treated with ReLeSR (Stem Cell Technologies) to dissociate into single cells, and cells suspended at 3×10⁴ cells/ml in STEMdiff APEL2 medium (Stem Cell Technologies), which is the medium composition for spin embryoid bodies, supplemented with 1× Penicillin-Streptomycin (Invitrogen), 40 ng/ml SCF (Invitrogen), 20 ng/ml VEGF (R&D), and 20 ng/ml BMP4 (R&D) were seeded at 3,000 cells per well in round-bottom 96-well plates, centrifuged at 1,500 rpm for 4 minutes, cultured in a 37 °C incubator for 3 to 4 days, and then half-medium exchanged with fresh culture medium and cultured for 9 to 11 days. On days 9 to 11 of spin embryoid body differentiation, spin embryoid bodies in Wells 6 to 8 of the 96-well plate were transferred to Well 1 of a 2% gelatin-coated 24-well plate, and cultured in differentiation culture medium comprising 85% DMEM/F12 (GIBCO), 15% FBS (GIBCO), 5 ng/ml sodium selenite (Sigma), 50 μM ethanolamine (Sigma), 20 μg/ml ascorbic acid (Sigma), 25 μM β-mercaptoethanol (GIBCO), 1× Glutamax (GIBCO), 1% Penicillin-Streptomycin (GIBCO), and the following cytokines: 5 ng/ml IL-3 (Peprotech), 10 ng/ml IL-15 (Peprotech), 20 ng/ml IL-7 (Peprotech), 20 ng/ml SCF (Invitrogen), and 10 ng/ml Flt3L (Peprotech). Every 5 to 7 days thereafter for 28 days, the cells were cultured with NK differentiation medium additionally supplemented with cytokines excluding IL-3 to obtain differentiated iPS-NK. Pure iPS-NK cells were isolated using an NK isolation kit (Miltenyi Biotec), and the iPS-NK cells subsequently cultured in culture medium containing 90% RPMI 1640, 10% FBS, 1% Penicillin-Streptomycin, 20 ng/ml IL-15, and 20 ng/ml IL-2 were used. The major cell populations were distinguished into the following NK cell phenotypes according to the expression of CD56 and CD16 markers: drNK: CD56^{superbright}CD16^{superbright}, NK92: CD56^{bright}CD16^{dim}, pNK (CD56^{dim}pNK): CD56^{dim}CD16^{bright}, Cytokine activated pNK (CD56^{bright}pNK): CD56^{bright}CD16^{bright}, iPS-NK: CD56^{bright}CD16^{dim} (FIGS. 2A and 2B). In particular, based on the fluorescence intensity of CD56 and CD16 in the most commonly used pNK (CD56^{dim}pNK), the levels were designated as dim (10⁴ or less), bright (10⁴ to 10⁵), and superbright (10⁵ or more).

### Example 3. Killing Ability of drNK Against Hepatic Fibrosis-Causing Human Hepatic Stellate Cells

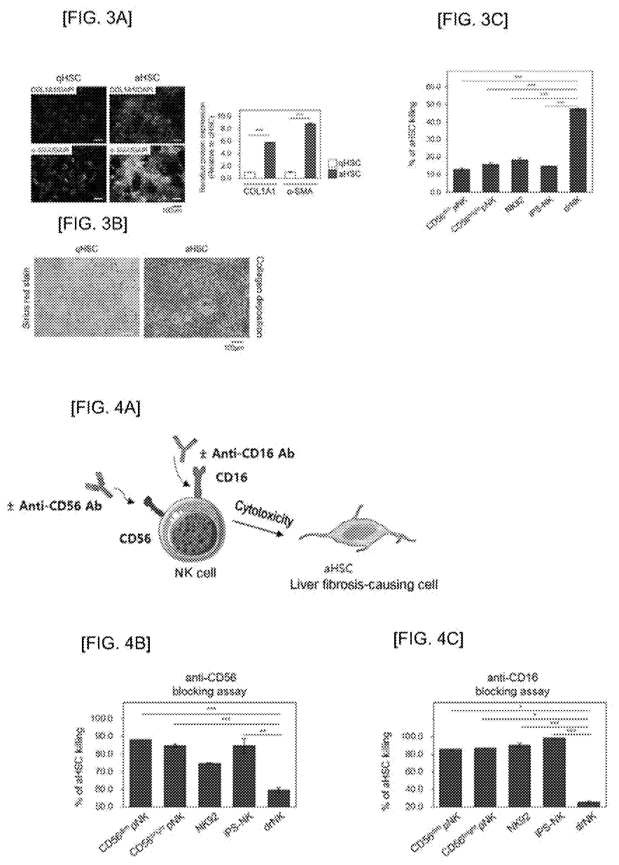

The LX-2 human hepatic stellate cell line was used as a cell model. Quiescent LX-2 cells (quiescent HSC; qHSC) were cultured in DMEM medium containing 2% FBS and treated with 20 ng/ml recombinant human TGF-β1 protein (rh TGF-β1 protein) for 48 hours to induce the activated state, thereby producing activated HSCs (aHSCs). Immunocytochemistry (ICC) was performed to confirm that the intracellular protein expression levels of skeletal proteins collagen (Collagen type I alpha 1, COL1A1) and α-smooth muscle actin (α-SMA), which develop in the extracellular matrix (ECM) that develops as fibrosis progresses in aHSCs, are high. When these results were converted into integrated density (IntDen) of fluorescence expression and quantified using Image J (NIH) program, COL1A1 expression increased by 5.77-fold and α-SMA expression increased by 8.83-fold in aHSCs compared to qHSCs, thereby verifying the significance as a hepatic fibrosis cell model (FIG. 3A).

In addition, examining the degree of collagen development using a Picrosirius red staining kit (Abcam) revealed that the density of red stain was higher compared to qHSC, indicating a higher degree of development into collagen connective tissue (FIG. 3B).

To determine the killing activity of NK cells against human hepatic stellate cells activated by TGF-β treatment (aHSCs), aHSCs were co-reacted with 2×10⁵ cells each of drNK and controls CD56^{dim} pNK, CD56^{bright} pNK, NK92, and iPS-NK cells at a 1:1 ratio in a 24-well plate. After 5 hours of reaction, propidium iodide (PI) staining was performed and analyzed by flow cytometry. As a result, the killing ability of the directly reprogrammed natural killer cells was confirmed to be superior relative to that of control groups (CD56^{dim} pNK, CD56^{bright} pNK, NK92, iPS-NK), by 3.63-fold compared to CD56^{dim} pNK, 2.98-fold compared to CD56^{bright} pNK, 2.60-fold compared to NK92, and 3.19-fold compared to iPS-NK (FIG. 3C).

### Example 4. Antifibrotic Effect of drNK

### Example 4-1. Antifibrotic effect of drNK Dependent on CD56 and CD16 Expression

To analyze the antifibrotic effect of drNK dependent on CD56 and CD16 expression, a cytotoxicity assay using anti-CD56 and anti-CD16 antibodies was performed (FIG. 4A). To analyze the direct correlation between aHSC cytotoxicity and CD56 expression, 2×10⁵ aHSC cells were reacted with 1 μg/ml anti-CD56 antibody for 30 minutes in a 24-well plate, and then the reacted aHSC were prepared by washing one or more times with culture medium. The prepared aHSC were co-reacted with 2×10⁵ cells each of drNK and controls CD56^{dim} pNK, CD56^{bright} pNK, NK92, and iPS-NK cells at 1:1 ratio for 5 hours, after which the cells were stained with propidium iodide (PI) and the killing ability was analyzed by flow cytometry. As a result, it was confirmed that the killing ability against anti-CD56-treated aHSC decreased relative to the anti-CD56-untreated control group (100%) for each cell type as follows: CD56^{dim} pNK (88.0%), CD56^{bright} pNK (84.64%), NK92 (74.86%), iPS-NK (84.69%), and drNK (59.57%). Through this, it was confirmed that the superior aHSC killing ability of the directly reprogrammed natural killer cells is most dependently associated with CD56 expression (FIG. 4B).

To analyze the direct correlation between aHSC cytotoxicity and CD16 expression, 2×10⁵ aHSC cells were reacted with 1 μg/ml anti-CD16 antibody for 30 minutes in a 24-well plate, and then the reacted aHSC were prepared by washing one or more times with culture medium. The prepared aHSC were co-reacted with 2×10⁵ cells each of drNK and controls CD56^{dim} pNK, CD56^{bright} pNK, NK92, and iPS-NK cells at 1:1 ratio for 5 hours, after which the cells were stained with propidium iodide (PI) and the killing ability was analyzed by flow cytometry. As a result, it was confirmed that the killing ability against anti-CD16-treated aHSC decreased relative to the anti-CD16-untreated control group (100%) for each cell type as follows: CD56^{dim} pNK (86.21%), CD56^{bright} pNK (87.42%), NK92 (90.23%), iPS-NK (98.38%), and drNK (25.37%). Through this, it was confirmed that the superior aHSC killing ability of the directly reprogrammed natural killer cells is most dependently associated with CD16 expression (FIG. 4C).

### Example 4-2. Antifibrotic Effect of drNK Dependent on NKG2D Expression

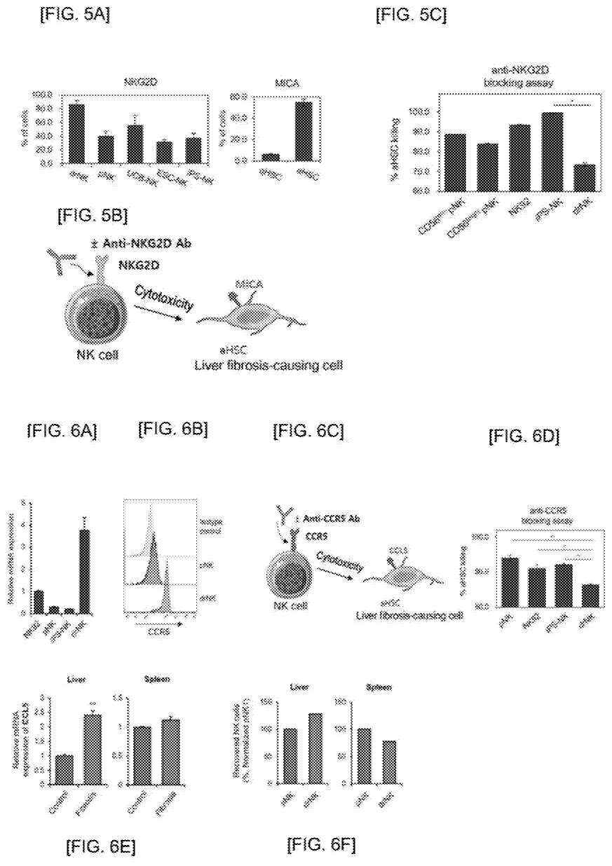

Analysis of NKG2D expression levels by flow cytometry revealed that the drNK of the present invention (86.20%) exhibits relatively higher NKG2D expression compared to known NK cells in the art: peripheral blood (pNK, 40.12%), placenta (UCB-NK, 55.33%), embryonic stem cell-derived (ESC-NK, 31.67%), and induced pluripotent stem cell-differentiated (iPS-NK, 36.67%) NK cells (FIG. 5A, left). In addition, higher NKG2D ligand MICA expression was observed in activated hepatic fibrogenic cells aHSC (54.5%) compared to control quiescent qHSC (6.4%) (FIG. 5A, right). To analyze the antifibrotic effect of drNK dependent on NKG2D expression, a cytotoxicity assay using an anti-NKG2D antibody was performed by utilizing the mechanism by which the NKG2D receptor of NK cells binds to the MICA ligand of aHSC (FIG. 5B). To analyze the direct correlation between aHSC cytotoxicity and NKG2D expression, 2×10⁵ aHSC cells were reacted with 1 μg/ml anti-NKG2D antibody for 30 minutes in a 24-well plate, and then the reacted aHSC were prepared by washing one or more times with culture medium. The prepared aHSC were co-reacted with 2×10⁵ cells each of drNK and controls CD56^{dim} pNK, CD56^{bright} pNK, NK92, and iPS-NK cells at 1:1 ratio for 5 hours, after which the cells were stained with propidium iodide (PI) and the killing ability was analyzed by flow cytometry. As a result, it was confirmed that the aHSC killing ability under anti-NKG2D-treated conditions decreased relative to the anti-NKG2D-untreated control group (100%) for each cell type as follows: CD56^{dim} pNK (88.74%), CD56^{bright} pNK (83.93%), NK92 (93.48%), iPS-NK (99.46%), drNK (73.33%), thereby confirming that the superior aHSC killing ability of the directly reprogrammed natural killer cells is most dependently associated with NKG2D expression (FIG. 5C).

### Example 4-3. Antifibrotic Effect of drNK Dependent on CCR5 Expression

RT-qPCR was performed to quantitatively analyze the expression of CCR5, the receptor gene for chemokine (C-C motif) receptor 5 [CCL5] known to increase in association with hepatic fibrosis, in drNK relative to control NK groups (NK-92, pNK, and iPS-NK cells). As a result, when the CCR5 gene expression level in NK-92 was set as the control value of 1, it was confirmed that the gene expression of CCR5 is relatively higher in drNK (3.75) compared to pNK (0.29) and iPS-NK (0.20) groups (FIG. 6A). Furthermore, upon labeling with an anti-CCR5 antibody and analysis by flow cytometry, it was confirmed that CCR5 is expressed at higher levels in drNK (93.4%) compared to control pNK (21.9%) (FIG. 6B).

To analyze the antifibrotic effect of drNK dependent on CCR5 expression, a cytotoxicity assay using an anti-CCR5 antibody was performed by utilizing the mechanism by which the CCR5 receptor of NK cells binds to the CCL5 ligand of aHSC, which are hepatic fibrosis-causing cells (FIG. 6C). To analyze the direct correlation between aHSC cytotoxicity and CCR5 expression, 2×10⁵ aHSC cells were reacted with 1 μg/ml anti-CCR5 antibody for 30 minutes in a 24-well plate, and then the reacted aHSC were prepared by washing one or more times with culture medium. The prepared aHSC were co-reacted with 2×10⁵ cells each of drNK and controls CD56^{dim} pNK, CD56^{bright} pNK, NK92, and iPS-NK cells at 1:1 ratio for 5 hours, after which the cells were stained with propidium iodide (PI) and the killing ability was analyzed by flow cytometry. As a result, it was confirmed that the aHSC killing ability under anti-CCR5-treated conditions decreased relative to the anti-CCR5-untreated control group (100%) for each cell type as follows: CD56^{dim} pNK (94.01%), CD56^{bright} pNK (82.13%), NK92 (90.99%), iPS-NK (92.21%), drNK (86.45%), thereby confirming that the superior aHSC killing ability of the directly reprogrammed natural killer cells is most dependently associated with CCR5 expression (FIG. 6D).

In the non-alcoholic steatohepatitis (NASH)-induced methionine-choline deficient (MCD)-fed mouse model of Example 8, blood and tissue analyses were performed to analyze the correlation between the antifibrotic effect of drNK and the CCL5-CCR5 axis. The experiment was conducted over a total of 9 weeks, with drNK administered at a dose of 1×10⁷ cells by intravenous injection (IV) at weeks 2 and 3 of feeding MCD diet. To analyze the cell distribution in mouse blood, blood was collected from the retro-orbital plexus. The liver and spleen of MCD mice were extracted, after which the tissues were lysed using a homogenizer and RNA was extracted, and the mRNA expression levels of each tissue were analyzed. When the expression level in the normal group (Control) was set as 1, the CCL5 expression levels were observed to be as follows: Control (1) and Fibrosis (2.4) in the liver, and Control (1) and Fibrosis (1.12) in the spleen (FIG. 6E).

In addition, to examine the residual pNK and drNK cells in each tissue, the liver and spleen of MCD mice were extracted, and then the tissues were lysed using a homogenizer, suspended in DPBS containing 1% BSA, and passed through a cell strainer to prepare a cell suspension. The cell suspension was reacted with ACK lysis buffer at 4 °C for 5 minutes, and then centrifuged at 800 G for 5 minutes. Subsequently, the cells were suspended in DPBS containing 0.5% BSA and 2 mM EDTA, stained with an anti-CD45 antibody, and analyzed by flow cytometry. As a result, it was observed that drNK remained 1.28-fold more than pNK in the liver, while drNK remained 0.78-fold less than pNK in the spleen (FIG. 6F). Through these results, it was confirmed that the infiltration of drNK into hepatic fibrosis tissue with increased CCL5 expression is greater compared to pNK, and that this is associated with the high CCR5 expression levels of drNK (FIG. 6F).

### Example 5. Therapeutic Effect on Hepatic Fibrosis

### Example 5-1. Superior Antifibrotic Effect of drNK in a Fibrotic Liver Organoid Model

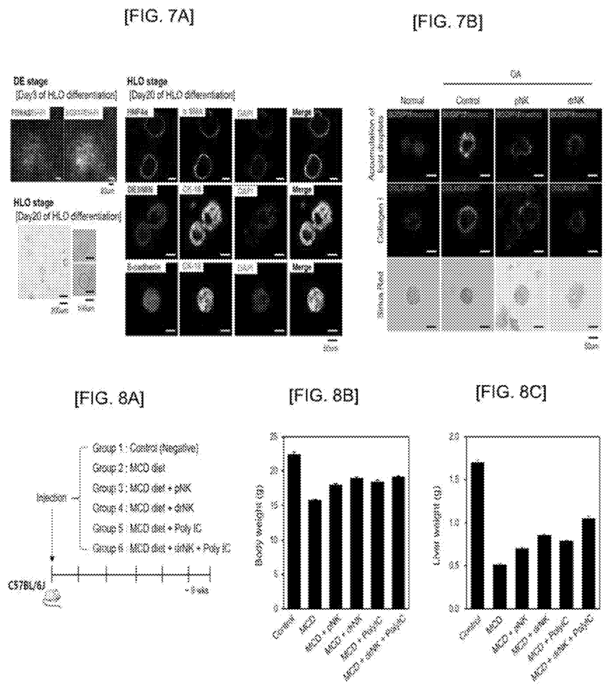

To analyze the antifibrotic effects of drNK, a human iPSC-derived multicellular liver organoid model simulating steatohepatitis was prepared. First, human iPSCs were differentiated into definitive endoderm in RPMI 1640 (Gibco) culture medium containing 50 ng/mL recombinant human bone morphogenetic protein-4 (hBMP-4) and 100 ng/mL recombinant human activin A. During this process, the serum concentration was gradually increased to D1: 0%, D2: 0.2%, and D3: 2%, and cultured for 3 days. Subsequently, the cells were cultured for additional 3 days in Advanced DMEM culture medium containing 500 ng/mL recombinant human fibroblast growth factor-4 (hFGF-4) and 3 μM CHIR99021 to form foregut spheroids, and successful induction of FOXA2 (1:100, anti-FOXA2, #07-633, Millipore) and SOX17 (1:50, anti-SOX17, #MAB1924, R&D) marker expression was confirmed by immunocytochemistry (FIG. 7A, left/upper). On day 6 of differentiation, Matrigel domes were prepared to embed the foregut spheroids, after which organoids were matured by culturing until day 10 in Advanced DMEM culture medium supplemented with 2 μM retinoic acid (RA). On day 10 of differentiation, the medium was replaced with William's E basal medium containing 10 ng/mL hepatocyte growth factor (HGF) and 20 ng/mL oncostatin M (OSM) supplemented with a hepatocyte maintenance supplement kit (cat. no. CM4000), and the organoids were cultured with medium replacement every 3 days until day 20 of differentiation (FIG. 7A, left/lower). Expression of HNF-4α (1:300, #3113s, Cell Signaling), α-SMA (1:20, #A5228, Sigma), Cytokeratin 18 (1:20, #M7010, DAKO), Desmin (1:50, #AB907, Chemicon), and E-cadherin (1:300, #3195, Cell Signaling) markers was confirmed by immunocytochemistry, and the formation of multicellular liver organoids composed of hepatocytes, hepatic epithelial cells, and stellate cells was confirmed, with a 3D structure approximately 100 μm in size (FIG. 7A, right).

Additionally, the prepared liver organoids were treated with 500 μM sodium oleate for 3 to 5 days to construct a fatty liver fibrosis model (Control) with accumulated fatty acids. Subsequently, the organoids were co-cultured with NK cells (direct reaction at a ratio of 1 organoid to 100 NK cells for 24 hours) to analyze the antifibrotic effect of drNK. Liver organoids cultured under NK cell (+/−) conditions were collected, fixed with 4% paraformaldehyde (PFA) for 2 hours at room temperature, washed with 1× DPBS, and centrifuged at 400 G for 3 minutes. Subsequently, the fixed organoids were infiltrated with 30% sucrose at 4 °C for one day, after which frozen blocks were prepared using an OCT compound, and sections of 15 μm thickness were prepared using a cryostat microtome (Sakura, Tissue-Tek TEC) for analysis. Lipid droplets were stained with BODIPY 493/503 fluorescent dye, cell nuclei were stained with Hoechst 33342, type 1 collagen protein was stained with an anti-COL1A1 antibody by immunocytochemistry, and cell nuclei were stained with DAPI. In addition, collagen tissues were stained using a Picrosirius red staining kit. As a result, it was confirmed that both the expression level of the hepatic fibrosis indicator BODIPY and collagen staining decreased in the groups treated with pNK and drNK cells compared to the fibrotic organoids (Control), thereby confirming the antifibrotic effect of NK cells (FIG. 7B). In particular, the recovery to normal levels was more pronounced in the group treated with drNK cells, thereby confirming the superior antifibrotic effect of drNK (FIG. 7B).

### Example 5-2. Antifibrotic Effects of drNK in MCD-Diet Mouse Model (Body Weight and Liver Weight)

The antifibrotic effect of natural killer cells was monitored over a total of 9 weeks in the NASH-induced MCD-diet mouse model. C57BL/6J mice (average weight of 20 g to 25 g) were used, and the experimental groups consisted of a total of 6 groups: Control, MCD alone, MCD+pNK, MCD+drNK, MCD+PolyIC, and MCD+drNK+PolyIC (FIG. 8A). In particular, pNK and drNK were each administered by intravenous injection (IV) at a dose of 1×10⁷ cells.

Upon measuring the body weight and liver weight of mice in each group over 9 weeks, the normal group (Control, approximately 22 g) exhibited a normal body weight increase, and in mice subjected to MCD feeding, body weight reduction was alleviated in the order of MCD+drNK+PolyIC (approximately 19 g), MCD+PolyIC (approximately 18 g), MCD+drNK (approximately 19 g), and MCD+pNK (approximately 18 g) compared to the MCD control group (approximately 16 g) (FIG. 8B). Through these results, it was confirmed that in the drNK cell group relative to the pNK group, the combined transplantation of drNK and Poly IC more effectively suppresses body weight reduction compared to single transplantation (FIG. 8B). In the case of liver weight, the values were analyzed as follows: Control (approximately 1.7 g), MCD (approximately 0.51 g), MCD+pNK (approximately 0.7 g), MCD+drNK (approximately 0.85 g), MCD+PolyIC (approximately 0.78 g), and MCD+drNK+PolyIC (approximately 1.05 g). Liver weight reduction was more effectively suppressed in the drNK cell group compared to the pNK cell group, and combined transplantation of drNK and Poly IC exhibited higher efficacy than single transplantation (FIG. 8C).

### Example 5-3. Antifibrotic Effect of drNK in MCD-Diet Mouse Model (Liver Damage Indicators and Steatosis)

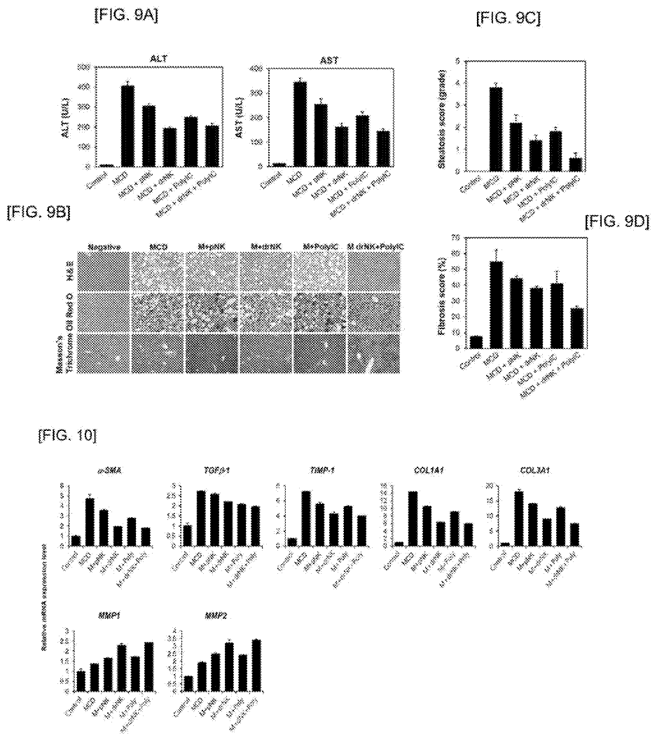

To confirm the antifibrotic effect of drNK through changes in liver damage indicator (ALT and AST) levels in the NASH-induced MCD-diet mouse model, blood samples were obtained from each group of mice by retro-orbital bleeding and analyzed. It was confirmed that ALT and AST levels increased in the MCD-fed group compared to the normal group (Control), and that liver enzyme levels relatively decreased in the groups that were administered NK cells and Poly IC compared to the group that was administered MCD diet alone [ALT group: Control (approximately 10 U/L), MCD alone (approximately 407 U/L), M+pNK (approximately 303.3 U/L), M+drNK (approximately 193 U/L), M+PolyIC (approximately 248.3 U/L), M+drNK+PolyIC (204.7 U/L); AST group: Control (approximately 11.33 U/L), MCD only (approximately 344.3 U/L), M+pNK (approximately 253.7 U/L), M+drNK (approximately 161.7 U/L), M+PolyIC (approximately 208.7 U/L), M+drNK+PolyIC (approximately 144.7 U/L)] (FIG. 9A). In particular, it was confirmed that drNK exhibited superior inhibitory effects on liver damage indicators compared to existing pNK, and that the effect was further enhanced upon combined administration of drNK and Poly IC (FIG. 9A). Hematoxylin & eosin (H&E) staining, Oil Red O staining, and Masson's trichrome staining were performed for histomorphological analysis (FIG. 9B).

From H&E staining, it was observed that the structure of the hepatic lobules was well maintained in the Control group with no morphological changes such as necrosis or inflammation; however, in the MCD-fed group, the hepatic structure was lost and numerous necrotic morphologies were observed. This phenomenon was alleviated in the groups that were administered NK cells and Poly IC. In particular, the inhibitory effect on structural loss was superior in the groups that were administered drNK cells compared to existing pNK, and it was confirmed that this effect was further enhanced upon combined administration of drNK cells and Poly IC.

From Oil Red O staining for assessing the degree of intracellular steatosis, lipid accumulation was more observed (red coloration) in the MCD-fed group compared to the Control group, and similarly to the H&E results, it was confirmed that the inhibitory effect on lipid accumulation was superior in the group transplanted with drNK cells compared to existing pNK, and that this effect was further enhanced upon combined administration of drNK cells and Poly IC. The degree of intracellular steatosis was evaluated on a 0 to 3 point scale, with asymptomatic cases scored as 0, mild lipid accumulation as 1, moderate accumulation as 2, and severe cases as 3, and the scores were summed for each group [Control (0 points), MCD alone (3.8 points), MCD+pNK (2.2 points), MCD+drNK (1.4 points), MCD+PolyIC (1.8 points), MCD+drNK+PolyIC (0.6 points)] (FIG. 9C). In addition, from measuring collagen development (fibrosis score) by Masson's trichrome staining, it was confirmed that drNK exhibited superior inhibitory effects on hepatic structural loss and steatosis compared to control pNK and Poly IC. The fibrosis score (%) was measured by converting the area of collagen development region to a ratio relative to the total image area based on the imaging results for each group after staining [Control (7.33%), MCD alone (54.9%), MCD+pNK (44.2%), MCD+drNK (38%), MCD+PolyIC (40.9%), MCD+drNK+PolyIC (25.2%)] (FIG. 9D). Accordingly, it was confirmed that drNK cells exhibit superior antifibrotic effects compared to pNK, such as amelioration of liver damage indicators and steatosis, and that a combined administration of drNK and Poly IC demonstrates a synergistic effect.

### Example 5-4. Antifibrotic Effects of drNK in MCD-Diet Mouse Model (Fibrosis Markers)

The antifibrotic effect of drNK in the NASH-induced MCD-diet mouse model was comparatively analyzed through the expression levels of fibrosis markers and extracellular matrix-degrading enzymes. RNA was extracted from the liver tissue of mice in each group at 9 weeks following NK cell administration, and RT-qPCR was performed. As a result, with Control group set as the reference value (1), the following fold-changes in marker expressions were confirmed: α-SMA: MCD alone (4.71-fold), M+pNK (3.55-fold), M+drNK (1.96-fold), M+PolyIC (2.77-fold), M+drNK+PolyIC (1.80-fold); TGF-β: MCD alone (2.73-fold), M+pNK (2.58-fold), M+drNK (2.20-fold), M+PolyIC (2.07-fold), M+drNK+PolyIC (1.95-fold); TIMP-1: MCD alone (7.24-fold), M+pNK (5.56-fold), M+drNK (4.30-fold), M+PolyIC (5.24-fold), M+drNK+PolyIC (3.99-fold); COL1A1: MCD alone (14.39-fold), M+pNK (10.56-fold), M+drNK (6.31-fold), M+PolyIC (9.10-fold), M+drNK+PolyIC (5.98-fold); COL3A1: MCD alone (18.00-fold), M+pNK (14.04-fold), M+drNK (8.99-fold), M+PolyIC (12.69-fold), M+drNK+PolyIC (7.39-fold); MMP-1: MCD alone (1.36-fold), M+pNK (1.65-fold), M+drNK (2.30-fold), M+PolyIC (1.71-fold), M+drNK+PolyIC (2.42-fold); MMP-2: MCD alone (1.91-fold), M+pNK (2.50-fold), M+drNK (3.20-fold), M+PolyIC (2.41-fold), M+drNK+PolyIC (3.40-fold) (FIG. 10). Through these results, it was verified that drNK more effectively suppresses the expression of fibrosis markers (α-SMA, TGF-β, TIMP-1, COL1A1, and COL3A1) and enhances the expression of extracellular matrix-degrading enzymes (MMP-1 and MMP-2) compared to pNK and PolyIC, thereby demonstrating a higher antifibrotic effect (FIG. 10).

### Example 5-5. Antifibrotic Effects of drNK in MCD-Diet Mouse Model (Inflammation)

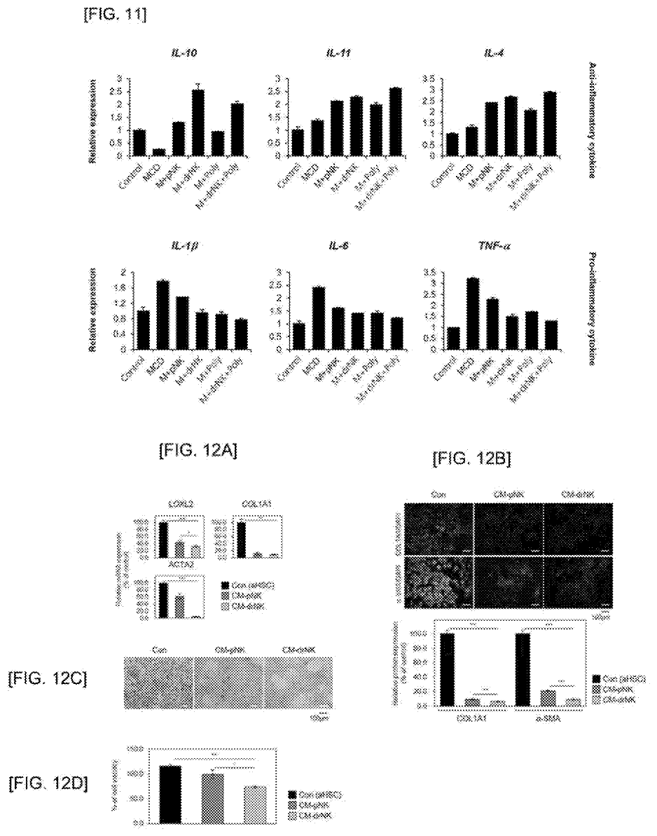

The antifibrotic effect of drNK in the NASH-induced MCD-diet mouse model was analyzed through the expression levels of anti-inflammatory and inflammatory markers. RNA was extracted from the liver tissues of mice in each group at 9 weeks following NK cell administration, and RT-qPCR was performed. As a result, with Control group set as the reference value (1), the following fold-changes in marker expressions were confirmed: IL-10: MCD alone (0.25-fold), M+pNK (1.30-fold), M+drNK (2.55-fold), M+PolyIC (0.94-fold), M+drNK+PolyIC (2.02-fold); IL-11: MCD alone (1.36-fold), M+pNK (2.12-fold), M+drNK (2.29-fold), M+PolyIC (1.98-fold), M+drNK+PolyIC (2.62-fold); IL-4: MCD alone (1.30-fold), M+pNK (2.41-fold), M+drNK (2.67-fold), M+PolyIC (2.07-fold), M+drNK+PolyIC (2.88-fold); IL-1β: MCD alone (1.77-fold), M+pNK (1.35-fold), M+drNK (0.95-fold), M+PolyIC (0.91-fold), M+drNK+PolyIC (0.77-fold); IL-6: MCD alone (2.40-fold), M+pNK (1.61-fold), M+drNK (1.40-fold), M+PolyIC (1.41-fold), M+drNK+PolyIC (1.22-fold); TNF-α: MCD alone (3.22-fold), M+pNK (2.28-fold), M+drNK (1.49-fold), M+PolyIC (1.71-fold), M+drNK+PolyIC (1.30-fold) (FIG. 11). Through these results, it was confirmed that drNK more effectively enhances the expression of anti-inflammatory markers (IL-10, IL-11, and IL-4) and exhibits superior inhibitory effects on the expression of inflammatory markers (IL-1β, IL-6, and TNF-α) compared to controls pNK and Poly IC (FIG. 11).

### Example 5-6. Inhibitory Effects of drNK-Conditioned Medium on the Activity of Fibrotic Human Hepatic Stellate Cells

To confirm the antifibrotic effects and actions of various proteins and cytokines secreted from NK cells, NK cells were cultured (2×10⁶ cells/ml) for 48 hours and conditioned medium (CM) was harvested, after which the effects on TGF-β1-mediated hepatic stellate cell activation were analyzed. When 5% NK-CM was added during TGF-β1-mediated hepatic stellate cell activation culture, it was confirmed by real-time polymerase chain reaction that the gene expression of fibrosis markers LOXL2 (CM-pNK: 43.72% and CM-drNK: 32.58%), COL1A1 (CM-pNK: 12.04% and CM-drNK: 9.76%), and ACTA2 (CM-pNK: 60.93% and CM-drNK: 4.34%) significantly decreased relative to 100% expression in aHSC (Con), and also that the effect of CM-drNK of the present invention is significantly superior compared to CM-pNK (FIG. 12A).

By immunocytochemistry, it was confirmed that the expression level of fibrosis marker proteins COL1A1 (CM-pNK: 9.26% and CM-drNK: 6.45%) and α-SMA (CM-pNK: 21.24% and CM-drNK: 9.45%) decreased to the level of qHSC relative to 100% expression in aHSC (Con) (FIG. 12B). Through Picrosirius red collagen staining, it was verified that the degree of red staining relative to aHSC (Con) further decreased in CM-drNK compared to CM-pNK, thereby confirming significant suppression of fibrosis (FIG. 12C). The EZ-Cytox kit (DoGenBio) was used to verify the viability (%) of fibrotic cells in the conditioned medium. During TGF-β1-mediated HSC fibrosis, tetrazolium was applied under conditions treated with CM-pNK or CM-drNK, and absorbance at 450 nm, which reacts with dehydrogenase in living cells, was measured. When qHSC was set as the reference value of 100%, it was confirmed that viability relative to aHSC (115.5%) further decreased under CM-drNK (73.6%) treatment conditions compared to control CM-pNK (99.0%) treatment conditions, thereby confirming that CM-drNK significantly suppresses conversion into fibrogenic cells (FIG. 12D).

### Example 6. Therapeutic Effect on Pulmonary Fibrosis

### Example 6-1. Superior Killing Ability of drNK against Fibrosis-Causing Cells in the Human Pulmonary Fibrosis Cell Model

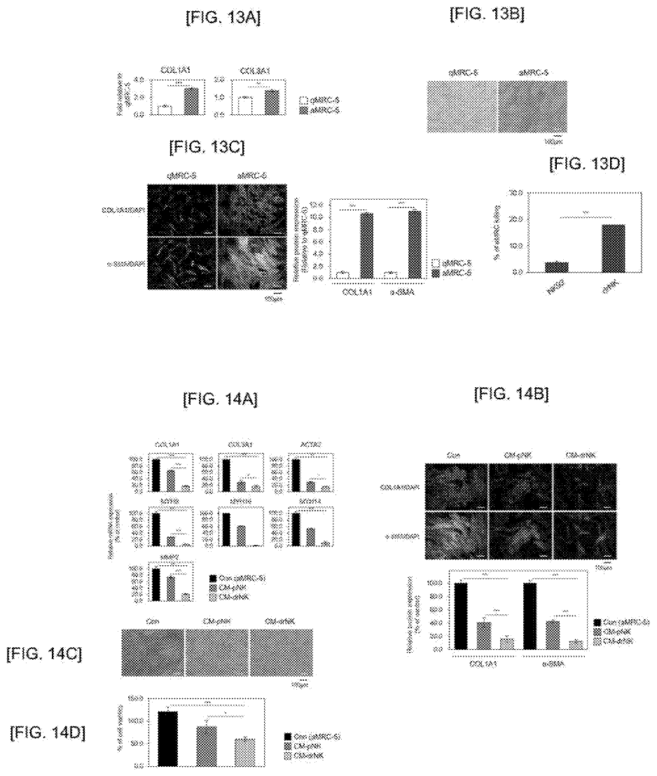

MRC-5, a human fetal lung fibroblast cell line, was used as the cell model. Quiescent MRC-5 (qMRC-5) cells were cultured in MEM medium containing 0.5% FBS, and fibrogenic activated pulmonary fibrotic cells (activated MRC-5; aMRC-5) were produced by treatment with 2 ng/ml recombinant human TGF-β1 protein (rh TGF-β1 protein) for 48 hours. It was confirmed by real-time polymerase chain reaction that the gene expression of fibrosis markers COL1A1 (2.98-fold) and COL3A1 (1.38-fold) significantly increased in aMRC-5 compared to qMRC-5 (FIG. 13A).

In addition, examining the degree of collagen development using a Picrosirius red staining kit (Abcam) revealed that the density of red stain was higher compared to qMRC-5, indicating a higher degree of development into collagen connective tissue (FIG. 13B). It was confirmed by immunocytochemistry that the expression levels of collagen (COL1A1) and α-SMA proteins increased as fibrosis progressed in aMRC-5. Quantification from integrated density (IntDen) of fluorescence expression levels using Image J (NIH) program revealed that the expression of COL1A1 and α-SMA was increased by 10.60-fold and 11.01-fold, respectively, in aMRC-5 compared to qMRC-5, thereby verifying the significance as a pulmonary fibrosis cell model (FIG. 13C).

To determine the killing activity of NK cells against human lung fibroblasts activated by TGF-β treatment (aMRC-5), aMRC-5 were co-reacted with 2×10⁵ cells each of drNK and control NK92 cells at 1:1 ratio in a 24-well plate. After 5 hours of reaction, propidium iodide (PI) staining was performed and analyzed by flow cytometry, which revealed that while control NK92 cells exhibited a killing ability of 3.7%, the directly reprogrammed natural killer cells exhibited 17.9%, demonstrating a 4.8-fold increase in direct killing ability (FIG. 13D).

### Example 6-2. Inhibitory Effects of drNK-Conditioned Medium on the Activity of Fibrotic Human Lung Fibroblasts

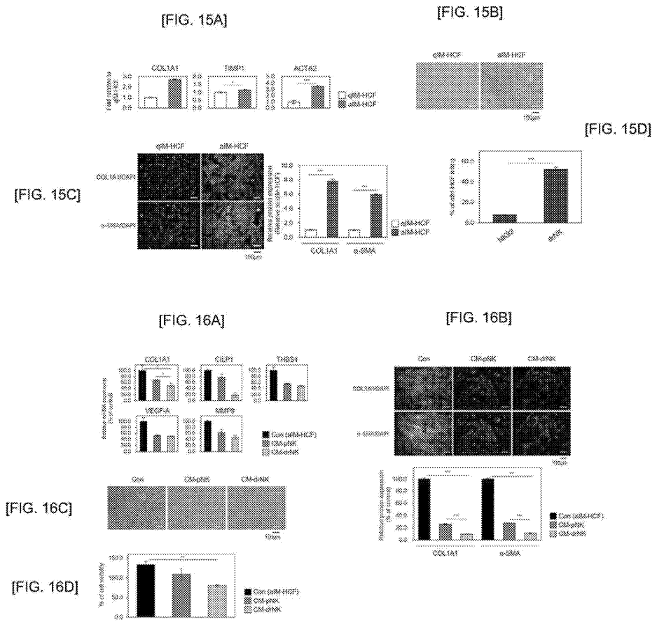

To confirm the antifibrotic effects and actions of various proteins and cytokines secreted from NK cells, NK cells were cultured (2×10⁶ cells/ml) for 48 hours and conditioned medium (CM) was harvested, after which the effects on TGF-β1-mediated lung fibroblast (MRC-5) activation were analyzed. When 5% NK-CM was added during TGF-β1-mediated MRC-5 activation culture, it was confirmed by real-time polymerase chain reaction that the gene expression of fibrosis markers COL1A1 (CM-pNK: 65.04% and CM-drNK: 18.08%), COL3A1 (CM-pNK: 29.93% and CM-drNK: 16.55%), ACTA2 (CM-pNK: 29.29% and CM-drNK: 16.02%), MYH9 (CM-pNK: 27.72% and CM-drNK: 5.04%), MYH10 (CM-pNK: 61.19% and CM-drNK: 1.67%), MYH14 (CM-pNK: 53.03% and CM-drNK: 9.14%), and MMP2 (CM-pNK: 73.88% and CM-drNK: 21.67%) significantly decreased relative to 100% expression in aMRC-5 (Con), and also that the effect of CM-drNK of the present invention is significantly superior compared to CM-pNK (FIG. 14A). By immunocytochemistry, it was confirmed that the expression level of fibrosis marker proteins COL1A1 (CM-pNK: 40.99% and CM-drNK: 16.00%) and α-SMA (CM-pNK: 42.54% and CM-drNK: 12.62%) decreased to the level of qMRC-5 relative to 100% expression in aMRC-5 (Con) (FIG. 14B).

Through Picrosirius red collagen staining, it was verified that the degree of red staining relative to aMRC-5 (Con) further decreased in CM-drNK compared to CM-pNK, thereby confirming significant suppression of fibrosis (FIG. 14C). The EZ-Cytox kit (DoGenBio) was used to verify the viability (%) of fibrotic cells in the conditioned medium. During TGF-β1-mediated MRC-5 fibrosis, tetrazolium was applied under conditions treated with CM-pNK or CM-drNK, and absorbance at 450 nm, which reacts with dehydrogenase in living cells, was measured. When qMRC-5 was set as the reference value of 100%, it was confirmed that viability relative to aMRC-5 (121.0%) further decreased under CM-drNK (59.8%) treatment conditions compared to control CM-pNK (87.0%) treatment conditions, thereby confirming that CM-drNK significantly suppresses conversion into fibrogenic cells (FIG. 14D).

### Example 7. Therapeutic Effect on Cardiac Fibrosis

### Example 7-1. Antifibrotic Effect of drNK in Cardiac Cell Model

IM-HCF (Immortalized human cardiac fibroblasts; P10453-IM), a human cardiac fibroblast cell line, was used as the cell model. Quiescent IM-HCF (qIM-HCF) cells were cultured in Fibroblast medium plus (FM-Plus) medium containing 5% FBS, and fibrogenic activated IM-HCF (aIM-HCF) were produced by treatment with 10 ng/ml recombinant human TGF-β1 protein (rh TGF-β1 protein) for 24 hours. It was established by real-time polymerase chain reaction that the gene expression of fibrosis markers COL1A1 (2.72-fold), TIMP1 (1.14-fold), and ACTA2 (3.39-fold) was significantly increased in aIM-HCF compared to qIM-HCF (FIG. 15A). In addition, examining the degree of collagen development using a Picrosirius red staining kit (Abcam) revealed that the density of red stain was higher compared to qIM-HCF, indicating a higher degree of development into collagen connective tissue (FIG. 15B).

It was confirmed by immunocytochemistry that the expression levels of collagen (COL1A1) and α-SMA proteins increased as fibrosis progressed in aIM-HCF. Quantification from integrated density (IntDen) of fluorescence expression levels using Image J (NIH) program revealed that the expression of COL1A1 and α-SMA was increased by 7.83-fold and 5.94-fold, respectively, in aIM-HCF compared to qIM-HCF, thereby verifying the significance as a cardiac fibrosis cell model (FIG. 15C). In addition, to determine the killing activity of NK cells against human cardiac fibroblasts activated by TGF-β treatment (aIM-HCF), aIM-HCF were co-reacted with 2×10⁵ cells each of drNK and control NK92 cells at 1:1 ratio in a 24-well plate. After 5 hours of reaction, propidium iodide (PI) staining was performed and analyzed by flow cytometry. As a result, it was confirmed that the directly reprogrammed natural killer cells (drNK) (52.3%) exhibit a 6.8-fold superior killing ability compared to control NK92 cells (7.6%) (FIG. 15D).

### Example 7-2. Inhibitory Effects of drNK-Conditioned Medium on the Activity of Fibrotic Human Cardiac Fibroblasts

To confirm the antifibrotic effects and actions of various proteins and cytokines secreted from NK cells, NK cells were cultured (2×10⁶ cells/ml) for 48 hours and conditioned medium (CM) was harvested, after which the effects on TGF-β1-mediated cardiac fibroblast (IM-HCF) activation were analyzed. When 5% NK-CM was added during TGF-β1-mediated IM-HCF activation culture, it was confirmed by real-time polymerase chain reaction that the gene expression of fibrosis markers COL1A1 (CM-pNK: 67.19% and CM-drNK: 51.35%), CILP1 (CM-pNK: 77.12% and CM-drNK: 18.47%), THBS4 (CM-pNK: 56.06% and CM-drNK: 48.72%), VEGF-A (CM-pNK: 52.78% and CM-drNK: 50.72%), and MMP9 (CM-pNK: 63.18% and CM-drNK: 46.75%) significantly decreased relative to 100% expression in aIM-HCF (Con), and also that the effect of CM-drNK of the present invention is significantly superior compared to CM-pNK (FIG. 16A).

By immunocytochemistry, it was confirmed that the expression level of fibrosis marker proteins COL1A1 (CM-pNK: 25.54% and CM-drNK: 9.60%) and α-SMA (CM-pNK: 27.88% and CM-drNK: 11.40%) decreased to the level of qIM-HCF relative to 100% expression in aIM-HCF (Con) (FIG. 16B). Through Picrosirius red collagen staining, it was confirmed that the degree of red staining relative to aIM-HCF (Con) was lower in CM-drNK compared to CM-pNK, thereby confirming significant suppression of fibrosis (FIG. 16C). The EZ-Cytox kit (DoGenBio) was used to verify the viability (%) of fibrotic cells in the conditioned medium. During TGF-β1-mediated IM-HCF fibrosis, tetrazolium was applied under conditions treated with CM-pNK or CM-drNK, and absorbance at 450 nm, which reacts with dehydrogenase in living cells, was measured. When qIM-HCF was set as the reference value of 100%, it was confirmed that viability relative to aIM-HCF (133.5%) was further decreased under CM-drNK (80.5%) treatment conditions compared to control CM-pNK (108.2%) treatment conditions, thereby confirming that CM-drNK significantly suppresses conversion into fibrogenic cells (FIG. 16D).

### Example 8. Therapeutic Effect on Dermal Fibrosis

### Example 8-1. Antifibrotic Effect of drNK in Skin Cell Model

HDF (Human foreskin dermal fibroblasts; CRL2097), a human dermal fibroblast cell line, was used as the cell model. Quiescent HDF (qHDF) cells were cultured in MEM medium containing 10% FBS, and fibrogenic activated HDF (aHDF) were produced by treatment with 20 ng/ml recombinant human TGF-β1 protein (rh TGF-β1 protein) for 48 hours. It was confirmed by real-time polymerase chain reaction that the gene expression of fibrosis markers COL1A1 (2.57-fold), COL3A1 (1.64-fold), and ACTA2 (2.59-fold) significantly increased in aHDF compared to qHDF (FIG. 17A). In addition, examining the degree of collagen development using a Picrosirius red staining kit (Abcam) revealed that the density of red stain was higher compared to qHDF, indicating a higher degree of development into collagen connective tissue (FIG. 17B).

It was confirmed by immunocytochemistry that the expression levels of collagen (COL1A1) and α-SMA proteins increased as fibrosis progressed in aHDF. Quantification from integrated density (IntDen) of fluorescence expression levels using Image J (NIH) program revealed that the expression of COL1A1 and α-SMA was increased by 13.89-fold and 6.18-fold, respectively, in aHDF compared to qHDF, thereby verifying the significance as a dermal fibrosis cell model (FIG. 17C).

To determine the killing activity of NK cells against human dermal fibroblasts activated by TGF-β treatment (aHDF), aHDF were co-reacted with 2×10⁵ cells each of drNK and control NK92 cells at a 1:1 ratio in a 24-well plate. After 5 hours of reaction, propidium iodide (PI) staining was performed and analyzed by flow cytometry. As a result, it was confirmed that the directly reprogrammed natural killer cells (drNK) (37.1%) exhibit a 1.6-fold superior killing ability compared to control NK92 cells (22.3%) (FIG. 17D).

### Example 8-2. Inhibitory Effects of drNK-Conditioned Medium on the Activity of Fibrotic Human Dermal Fibroblasts

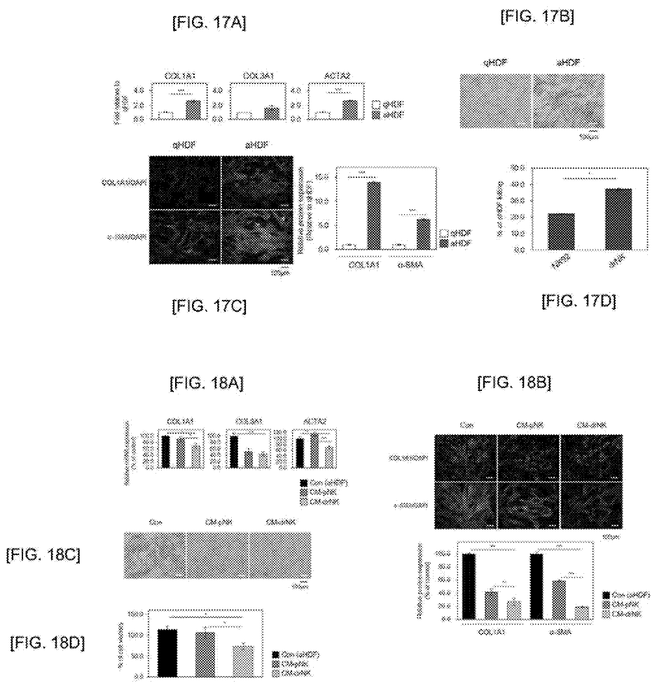

To confirm the antifibrotic effects and actions of various proteins and cytokines secreted from NK cells, NK cells were cultured (2×10⁶ cells/ml) for 48 hours and conditioned medium (CM) was harvested, after which the effects on TGF-β1-mediated dermal fibroblast (HDF) activation were analyzed. When 5% NK-CM was added during TGF-β1-mediated dermal HDF activation culture, it was confirmed by real-time polymerase chain reaction that the gene expression of fibrosis markers COL1A1 (CM-pNK: 90.66% and CM-drNK: 69.55%), COL3A1 (CM-pNK: 50.67% and CM-drNK: 43.20%), and ACTA2 (CM-pNK: 114.89% and CM-drNK: 70.24%) significantly decreased relative to 100% expression in aHDF (Con), and also that the effect of CM-drNK of the present invention is significantly superior compared to CM-pNK (FIG. 18A).

By immunocytochemistry, it was confirmed that the expression level of fibrosis marker proteins COL1A1 (CM-pNK: 41.44% and CM-drNK: 26.43%) and α-SMA (CM-pNK: 58.48% and CM-drNK: 18.69%) decreased to the level of qHDF relative to 100% expression in aHDF (Con) (FIG. 18B).

Through Picrosirius red collagen staining, it was verified that the degree of red staining relative to aHDF (Con) further decreased in CM-drNK compared to CM-pNK, thereby confirming significant suppression of fibrosis (FIG. 18C). The EZ-Cytox kit (DoGenBio) was used to verify the viability (%) of fibrotic cells in the conditioned medium. During TGF-β1-mediated HDF fibrosis, tetrazolium was applied under conditions treated with CM-pNK or CM-drNK, and absorbance at 450 nm, which reacts with dehydrogenase in living cells, was measured. When qHDF was set as the reference value of 100%, it was confirmed that viability relative to aHDF (113.6%) further decreased under CM-drNK (73.5%) treatment conditions compared to control CM-pNK (106.1%) treatment conditions, thereby confirming that CM-drNK significantly suppresses conversion into fibrogenic cells (FIG. 18D).

### Example 9. Therapeutic Effect on Renal Fibrosis

### Example 9-1. Antifibrotic Effect of drNK in Renal Cell Model

HK-2 (Human immortalized proximal tubule epithelial cell), a human renal tubular epithelial cell line, was used as the cell model. Quiescent HK-2 (qHK-2) cells were cultured in RPMI 1640 medium containing 10% FBS, and fibrogenic activated renal fibrotic cells (activated HK-2; aHK-2) were produced by treatment with 10 ng/ml recombinant human TGF-β1 protein (rh TGF-β1 protein) for 48 hours. It was confirmed by real-time polymerase chain reaction that the gene expression of fibrosis markers COL1A1 (8.06-fold), COL3A1 (5.54-fold), and ACTA2 (4.76-fold) significantly increased in aHK-2 compared to qHK-2, and the fibrogenic activated cells aHK-2 were established (FIG. 19A). In addition, examining the degree of collagen development using a Picrosirius red staining kit (Abcam) revealed that the density of red stain was higher compared to qHK-2, indicating a higher degree of development into collagen connective tissue (FIG. 19B).

It was confirmed by immunocytochemistry that the expression levels of collagen (COL1A1) and α-SMA proteins increased as fibrosis progressed in aHK-2. Quantification from integrated density (IntDen) of fluorescence expression levels using Image J (NIH) program revealed that the expression of COL1A1 and α-SMA was increased by 8.94-fold and 3.77-fold, respectively, in aHK-2 compared to qHK-2, thereby verifying the significance as a renal fibrosis cell model (FIG. 19C).

To determine the killing activity of NK cells against human renal fibroblasts activated by TGF-β treatment (aHK-2), aHK-2 were co-reacted with 2×10⁵ cells each of drNK and control NK92 cells at 1:1 ratio in a 24-well plate. After 5 hours of reaction, propidium iodide (PI) staining was performed and analyzed by flow cytometry, which revealed that while control NK92 cells exhibited a killing ability of 17.1%, the directly reprogrammed natural killer cells exhibited 27.9%, demonstrating a 1.6-fold increase in direct killing ability (FIG. 19D).

### Example 9-2. Inhibitory Effects of drNK-Conditioned Medium on the Activity of Fibrotic Human Renal Tubular Epithelial Cells

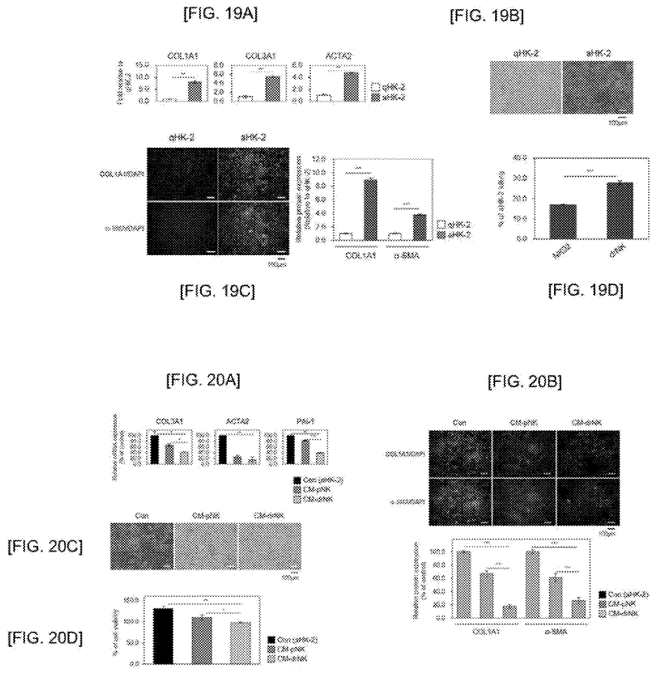

To confirm the antifibrotic effects and actions of various proteins and cytokines secreted from NK cells, NK cells were cultured (2×10⁶ cells/ml) for 48 hours and conditioned medium (CM) was harvested, after which the effects on TGF-β1-mediated renal tubular epithelial cell (HK-2) activation were analyzed. When 5% NK-CM was added during TGF-β1-mediated renal HK-2 activation culture, it was confirmed by real-time polymerase chain reaction that the gene expression of fibrosis markers COL3A1 (CM-pNK: 65.26% and CM-drNK: 41.74%), ACTA2 (CM-pNK: 25.48% and CM-drNK: 15.33%), and PAI-1 (CM-pNK: 82.27% and CM-drNK: 39.72%) significantly decreased relative to 100% expression in aHK-2 (Con), and also that the effect of CM-drNK of the present invention is significantly superior compared to CM-pNK (FIG. 20A).

By immunocytochemistry, it was confirmed that the expression level of fibrosis marker proteins COL1A1 (CM-pNK: 66.69% and CM-drNK: 17.41%) and α-SMA (CM-pNK: 61.15% and CM-drNK: 26.65%) decreased to the level of qHK-2 relative to 100% expression in aHK-2 (Con) (FIG. 20B).

Through Picrosirius red collagen staining, it was verified that the degree of red staining relative to aHK-2 (Con) further decreased in CM-drNK compared to CM-pNK, thereby confirming significant suppression of fibrosis (FIG. 20C). The EZ-Cytox kit (DoGenBio) was used to verify the viability (%) of fibrotic cells in the conditioned medium. During TGF-β1-mediated HK-2 fibrosis, tetrazolium was applied under conditions treated with CM-pNK or CM-drNK, and absorbance at 450 nm, which reacts with dehydrogenase in living cells, was measured. When qHK-2 was set as the reference value of 100%, it was confirmed that viability relative to aHK-2 (130.7%) further decreased under CM-drNK (97.9%) treatment conditions compared to control CM-pNK (110.0%) treatment conditions, thereby confirming that CM-drNK significantly suppresses conversion into fibrogenic cells (FIG. 20D).

### Example 10. Quantitative Analysis of Cytokine/Chemokine Gene Expression Levels in drNK

qRT-PCR was performed on the cytokine/chemokine gene groups in drNK to identify genes exhibiting distinctive expression patterns compared to control NKs (NK92 and iPS-NK cells).

As a result, ten genes (CCL5, IFN-γ, CXCL11, CXCL12, GDNF, VEGF, XCL1, IL16, LIF, and LTB) that were relatively upregulated in drNK compared to the NK92 and iPS-NK groups were identified. When the expression level in NK92 was set as the reference value of 1, the fold expression in iPS-NK and drNK was as follows: CCL5: NK92 (1), iPS-NK (0.85), drNK (3.64); IFN-γ: NK92 (1), iPS-NK (0.1), drNK (1.9); CXCL11: NK92 (1), iPS-NK (0.01), drNK (2.93); CXCL12: NK92 (1), iPS-NK (1.82), drNK (5.73); GDNF: NK92 (1), iPS-NK (0.03), drNK (5.69); VEGF: NK92 (1), iPS-NK (1.29), drNK (2.3); XCL1: NK92 (1), iPS-NK (0.18), drNK (2.15); IL16: NK92 (1), iPS-NK (0.98), drNK (4.35); LIF: NK92 (1), iPS-NK (1.78), drNK (8.5); and LTB: NK92 (1), iPS-NK (1.02), drNK (2.01) (FIG. 21).

### Example 11. Identification of drNK Secretory Factors Using a Human Proteome Cytokine Array

Analysis of the drNK-conditioned medium was performed to identify cell-secreted substances, particularly cytokines, that may influence the antifibrotic effect of drNK. Specifically, to obtain conditioned medium (CM) from NK cells, drNK cells and CD56^{dim}pNK cells were each cultured at a density of 10⁶ cells/ml. After 24 hours, the cultured medium was filtered through a 0.22 μm filter (Millipore). To measure cytokine levels in CM-drNK and CM-CD56^{dim}pNK, a proteome profiler array (Proteome Profiler Human XL Cytokine Array Kit, ARY022B; R&D Systems) was used according to the manufacturer's instructions, and quantitative analysis of the final images was performed using ImageJ software.

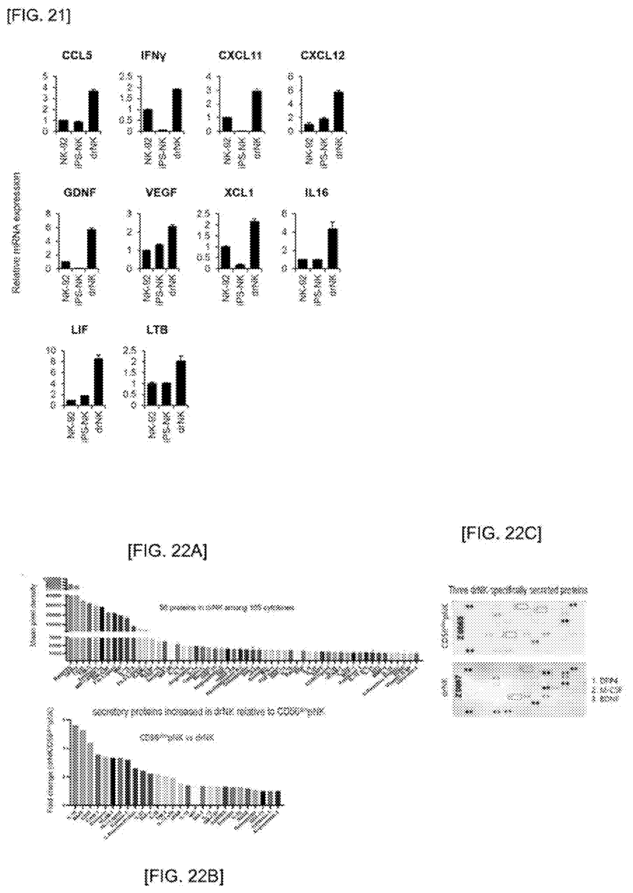

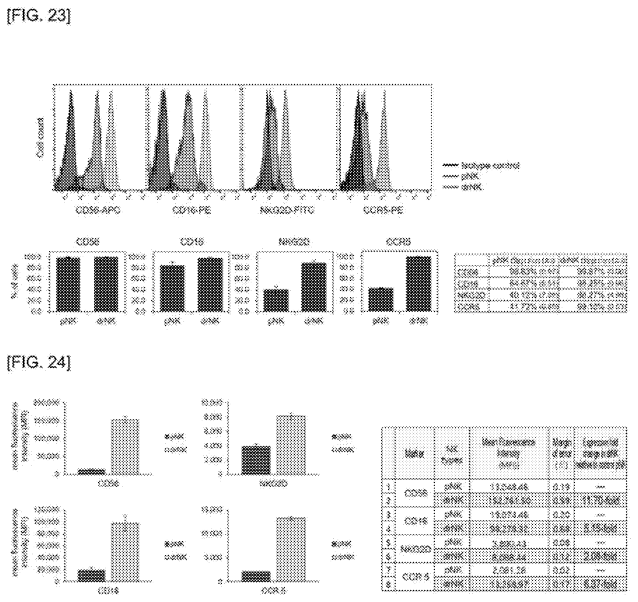

Using ImageJ, 56 drNK secretory proteins with fluorescence expression levels (mean pixel density, MPD) of 1,000 or above were identified, and the following major proteins were identified: RANTES (mean 81,174 MPD), DPPIV (mean 78,046 MPD), CD31 (mean 33,901 MPD), TIM-3 (mean 31,688 MPD), Emmprin (mean 29,204 MPD), MIP-1α/MIP-1β (mean 27,937 MPD), GM-CSF (mean 22,299 MPD), Fas Ligand (mean 21,602 MPD), MIF (mean 19,398 MPD), IL-16 (mean 16,867 MPD), IL-17A (mean 8,249 MPD), Flt-3 Ligand (mean 5,834 MPD), ICAM-1 (mean 4,532 MPD), M-CSF (mean 2,867 MPD), FGF-19 (mean 2,481 MPD), Serpin E1, MIP-3β (mean 2,165 MPD), IL-18 (mean 2,144 MPD), IL-32 (mean 1,950 MPD), Angiogenin (mean 1,901 MPD), IL-1α (mean 1,895 MPD), Cystatin C (mean 1,792 MPD), Resistin (mean 1,699 MPD), GDF-15 (mean 1,635 MPD), Angiopoietin-2 (mean 1,558 MPD), TNF-α (mean 1,546 MPD), PDGF-AA (mean 1,536 MPD), Apolipoprotein A-I (mean 1,507 MPD), Osteopontin (mean 1,489 MPD), Dkk-1 (mean 1,447 MPD), uPAR (mean 1,422 MPD), Endoglin (mean 1,411 MPD), IFN-γ (mean 1,394 MPD), FGF basic (mean 1,381 MPD), SDF-1α (CXCL12) (mean 1,378 MPD), IL-1β (mean 1,363 MPD), RAGE (mean 1,327 MPD), EGF (mean 1,277 MPD), IL-8 (mean 1,252 MPD), IL-27 (mean 1,208 MPD), BAFF (mean 1,191 MPD), CD40 ligand (mean 1,159 MPD), IL-34 (mean 1,129 MPD), VCAM-1 (mean 1,127 MPD), IL-1ra (mean 1,113 MPD), MCP-1 (mean 1,094 MPD), Kallikrein 3 (mean 1,088 MPD), IL-12 p70 (mean 1,088 MPD), IL-11 (mean 1,087 MPD), ST2 (mean 1,059 MPD), MMP-9 (mean 1,058 MPD), IL-22 (mean 1,057 MPD), C-Reactive Protein (mean 1,041 MPD), BDNF (mean 1,034 MPD), Vitamin D BP (mean 1,032 MPD), and Lipocalin-2 (mean 1,004 MPD) (FIG. 22A).

It was confirmed that 28 proteins were secreted at quantitatively higher levels in drNK compared to control CD56^{dim}pNK, and the following major proteins were identified: IL-16 (5.58-fold), BAFF (5.23-fold), CD31 (4.36-fold), ICAM-1 (3.57-fold), Emmprin (3.39-fold), VCAM-1 (3.33-fold), Flt-3 Ligand (3.32-fold), Cystatin C (3.16-fold), C-Reactive Protein (2.58-fold), IL-27 (2.44-fold), TNF-α (2.22-fold), IL-32 (2.16-fold), TIM-3 (2.01-fold), IL-12 p70 (1.94-fold), uPAR (1.53-fold), IL-18 Bpa (1.40-fold), MIF (1.39-fold), Dkk-1 (1.35-fold), IL-11 (1.33-fold), GM-CSF (1.30-fold), RANTES (1.29-fold), Endoglin (1.24-fold), IL-22 (1.16-fold), RAGE, Osteopontin (1.06-fold), GDF-15 (1.02-fold), Kallikrein 3 (1.02-fold), and Angiopoietin-2 (1.02-fold) (FIG. 22B). In addition, three drNK-specific proteins, namely DPP4, M-CSF, and BDNF, were identified (FIG. 22C).

### Example 12. Quantitative Analysis of Homogeneity and Superbright Phenotype of drNK Cells

In this example, the expression ratio and expression intensity of surface markers on drNK cells were analyzed in comparison with pNK.

As shown in FIG. 23, drNK was confirmed to be a homogeneous cell population with an increased proportion of cells simultaneously expressing CD56, CD16, NKG2D, and CCR5 compared to pNK. In particular, the proportion of CD16, NKG2D, and CCR5-positive cells was significantly higher in drNK compared to pNK, and it was observed that CCR5 is expressed in the majority of drNK cells. In the case of CCR5, expression was observed in approximately 99% or more of the cells. Furthermore, as shown in FIG. 24, analysis of the mean fluorescence intensity (MFI) of each surface marker revealed that the expression intensity of CD56, CD16, and CCR5 in drNK was significantly increased compared to pNK. drNK exhibited approximately 11.7-fold, 5.1-fold, and 6.3-fold increased expression of CD56, CD16, and CCR 5, respectively, compared to pNK (FIG. 24). In the present invention, “superbright” is defined as a state exhibiting MFI that is increased by at least 2-fold, specifically at least 5-fold, compared to control NK cells. These results indicate that drNK is a highly functional cell population having a quantitatively distinct “superbright” phenotype, rather than NK cells simply expressing these markers.

Accordingly, the drNK of the present invention is a homogeneous cell population amenable to quality control based on the expression ratio and expression intensity of multiple surface markers including CD56, CD16, NKG2D, and CCR5, which is useful in the manufacture of cell therapy agent for ensuring inter-batch consistency and predictability of therapeutic efficacy.

### Example 13. Analysis of Antifibrotic Mechanism of drNK Via Chemokine Ligand-Receptor Axis

### Example 13.1. Analysis of Expression of Chemokine and Fibrosis-Related Factors in Fibrotic Cells

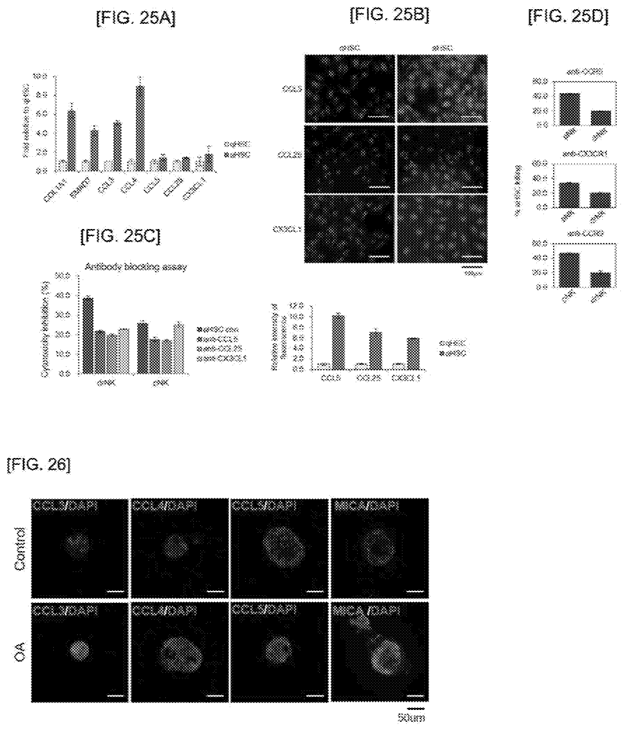

In this example, it was analyzed whether the interaction between chemokine ligands expressed on fibrotic cells and the receptors of drNK is involved in the antifibrotic function of drNK. As shown in FIG. 25A, activated HSC (aHSC) exhibited increased expression of fibrosis-related genes such as COL1A1 and SMAD7 compared to quiescent HSC (qHSC), and the expression of chemokine ligands including CCL3, CCL4, CCL5, CCL25, and CX3CL1 was also increased. This indicates that ligands corresponding to NK cell chemokine receptors (CCR5, CCR9, CX3CR1, etc.) are selectively overexpressed in fibrotic cells.

### Example 13.2. Analysis of Chemokine Ligand Protein Expression in Fibrotic Cells

In this example, the protein expression level of chemokine ligands in qHSC and aHSC was analyzed through fluorescence imaging. As shown in FIG. 25B, while the expression of CCL5, CCL25, and CX3CL1 was observed at low levels in qHSC, these ligands were strongly expressed throughout the cells in aHSC. Furthermore, quantitative analysis of fluorescence intensity revealed that the ligand expression levels in aHSC were significantly increased compared to qHSC. This indicates that fibrotic cells acquire molecular characteristics that can be recognized by NK cells.

### Example 13.3. Analysis of Changes in Antifibrotic Function of drNK Following Blocking of Chemokine Ligands

In this example, antibody blocking experiments were performed to evaluate the effect of chemokine ligands on the antifibrotic function of drNK. As shown in FIG. 25C, when treated with neutralizing antibodies against CCL5, CCL25, and CX3CL1, the cytotoxicity of drNK against aHSC was significantly reduced. In particular, this reduction was more pronounced in drNK compared to pNK. This indicates that the antifibrotic effect of drNK is dependent on interactions with multiple chemokine ligands rather than a single ligand, suggesting a multi-ligand-based mechanism of action.

### Example 13.4. Analysis of the Functional Dependence of drNK Through Blocking of Chemokine Receptors

In this example, it was evaluated whether the antifibrotic function of drNK depends on specific chemokine receptors. As shown in FIG. 25D, when treated with antibodies blocking CCR5, CX3CR1, and CCR9 receptors, the killing ability of drNK against aHSC was significantly decreased. In particular, the decrease rate in drNK was greater compared to pNK, which implies that drNK exhibits a relatively higher functional dependence on these receptors. These results demonstrate that drNK selectively eliminates fibrotic cells through a multi-step mechanism of homing via CCR5, cell adhesion via CX3CR1, tissue selectivity (targeting) via CCR9, and cell killing via NKG2D.

### Example 14. Antifibrotic Effect in a Three-Dimensional Organoid Model

In this example, changes in expression of ligands related to the antifibrotic action of drNK were analyzed using a three-dimensional fibrotic organoid model. As shown in FIG. 26, upon comparison of a normal organoid (control) and a fibrotic organoid (OA), the expression of chemokine ligands such as CCL3, CCL4, and CCL5, as well as MICA, an NKG2D-recognizing ligand, increased under OA conditions. The above ligands provide a molecular basis for interaction with the chemokine receptors or activation receptors of drNK. These results demonstrate that the level of ligands enabling drNK to recognize target cells are increased in a fibrotic tissue-like environment, suggesting that the antifibrotic effect of drNK is not limited to two-dimensional cell culture conditions and can also be applied to three-dimensional tissue-like environments.

In summary, it was confirmed that the directly reprogrammed natural killer cells of the present invention exhibit a superior direct killing ability compared to control natural killer cells known in the art when treated against hepatic stellate cells (hepatic fibrosis cell model), alveolar epithelial cells (pulmonary fibrosis cell model), cardiac cells (cardiac fibrosis cell model), skin cells (dermal fibrosis cell model), and renal cells (renal fibrosis cell model), which are fibrosis-causing cells that induce fibrotic responses; furthermore, when conditioned medium from the directly reprogrammed natural killer cells was applied to the fibrosis cell models above, collagen production, activity, and viability thereof were significantly reduced compared to that from control natural killer cells known in the art, thereby confirming an excellent efficacy in suppressing fibrosis, which suggests that fibrotic diseases can be prevented or treated.

Based on the above description, it will be understood by those skilled in the art that the present invention may be implemented in a different specific form without changing the technical spirit or essential characteristics thereof. In this regard, it should be understood that the foregoing examples are illustrative in all respects and are not to be construed as limiting. The scope of the present invention should be interpreted to include all modifications or modified forms derived from the meaning and scope of the claims as set forth below, as well as equivalent concepts, rather than from the detailed description above.

1. A pharmaceutical composition for preventing or treating fibrosis comprising directly reprogrammed natural killer cells (drNK cells),
wherein the drNK cells express one or more selected from the group consisting of CD56^{superbright}, CD16^{superbright}, and a combination thereof, and wherein “superbright” means that the mean fluorescence intensity (MFI) of CD56 or CD16 is at least 2-fold greater compared to that of a control natural killer cell measured under the same conditions.

2. The pharmaceutical composition according to claim 1, wherein the drNK cells exhibit cytotoxicity against fibrotic cells via NKG2D or CCR5 receptors, and decrease in cytotoxicity when the receptors are blocked.

3. The pharmaceutical composition according to claim 1, wherein the drNK cells further express NKG2D or CCR5.

4. The pharmaceutical composition according to claim 1, wherein the drNK cells express one or more factors selected from the group consisting of CCL5, IFN-γ, CXCL11, CXCL12, GDNF, VEGF, XCL1, IL16, LIF, and LTB.

5. The pharmaceutical composition according to claim 1, wherein the drNK cells express one or more proteins selected from the group consisting of DDP4, M-CSF, and BDNF.

6. The pharmaceutical composition according to claim 1, wherein the drNK cells are prepared by a method comprising:
(a) introducing a reprogramming factor into isolated cells; and
(b) culturing the same in a medium comprising cytokines, growth factors, a glycogen synthase kinase 3β (GSK3β) inhibitor, and an aryl hydrocarbon receptor (AHR) inhibitor to produce natural killer cells.

7. The pharmaceutical composition according to claim 6, wherein the isolated cells are somatic cells excluding natural killer cells.

8. The pharmaceutical composition according to claim 1, wherein the fibrosis develops from one or more selected from the group consisting of lung, kidney, liver, heart, brain, blood vessels, joints, intestine, skin, soft tissue, bone marrow, penis, peritoneum, muscle, spine, testis, ovary, breast, thyroid, tympanic membrane, pancreas, gallbladder, bladder, prostate, esophagus, stomach, uterus, spleen, lymph nodes, salivary glands, and nervous system.

9. A cell therapy agent for preventing or treating fibrosis comprising directly reprogrammed natural killer cells (drNK),
wherein the drNK cells express one or more selected from the group consisting of CD56^{superbright}, CD16^{superbright}, and a combination thereof,
wherein “superbright” means that the mean fluorescence intensity (MFI) of CD56 or CD16 is at least 2-fold greater compared to that of a control natural killer cell measured under the same conditions, and
wherein the cell therapy agent is provided as a cryopreserved formulation, an injectable formulation, or a formulation for intravenous administration.

10. A method for preventing or treating fibrosis, comprising administering a therapeutically effective amount of the pharmaceutical composition of claim 1 to a subject having fibrosis or at risk of fibrosis.
